(19) United States
(12) Patent Application Publication
(10) Pub. No.: US 2007/0161611 A1
(43) Pub. Date: Jul. 12, 2007

(54) POLYCYCLIC PHENOLIC COMPOUNDS AND USE IN TREATING VIRAL INFECTIONS

(76) Inventor: Dominique Dugourd, Vancouver (CA)

Correspondence Address: DARBY & DARBY P.C., P. O. BOX 5257, NEW YORK, NY, 10150-5257, US

(21) Appl. No.: 11/565,621

(22) Filed: Nov. 30, 2006

Related U.S. Application Data
(60) Provisional application No. 60/742,058, filed on Dec. 1, 2005.

(51) Int. Cl. A61K 31/56; A61K 31/58

(52) U.S. Cl. 514/176; 514/182; 514/178

(57) ABSTRACT

The present invention provides antiviral polycyclic phenolic compounds (PPCs) for use in treating or preventing viral infections and associated conditions, such as infections by Flaviviridae, Hepadnaviridae, Herpesviridae, Papillomaviridae, Retroviridae, Adenoviridae, or respiratory viruses (such as Adenoviridae, Orthomyxoviridae, Paramyxoviridae and Coronaviridae).

### CROSS-REFERENCE TO RELATED APPLICATION

This application claims the benefit of U.S. Provisional Application No. 60/742,058, filed Dec. 1, 2005, which application is incorporated by reference in it's entirety.

### TECHNICAL FIELD

The present invention relates generally to anti-infective polycyclic phenolic compounds (PPCs) and, in particular, to tetracyclic (steroid-like) compounds for use in treating or preventing viral infections and associated conditions, such as infections by Flaviviridae, Hepadnaviridae, Herpesviridae, Papillomaviridae, Retroviridae, Adenoviridae, or respiratory viruses (such as Adenoviridae, Orthomyxoviridae, Paramyxoviridae and Coronaviridae).

### BACKGROUND

The estrogen steroid hormones (e.g., 17-β-estradiol, estrone) and structurally related derivative compounds have attracted considerable interest as candidate cytoprotectants for use in the treatment of degenerative disorders and, in particular, as neuroprotectants based on a number of chemical and biological properties (see, e.g., U.S. Pat. Nos. 4,897,389, U.S. Pat. No. 5,512,557, U.S. Pat. No. 5,554,601, U.S. Pat. No. 5,554,603, U.S. Pat. No. 5,824,672, U.S. Pat. No. U.S. 5,843,934, U.S. Pat. No. 5,859,001, U.S. Pat. No. 5,866,561, U.S. Pat. No. 5,877,169, U.S. Pat. No. 5,972923, U.S. Pat. No. 5,990,177, U.S. Pat. No. 6,172,056, U.S. Pat. No. 6,172,088, U.S. Pat. No. U.S. 6,197,833, U.S. Pat. No. 6,207,658, U.S. Pat. No. 6,232,326, U.S. Pat. No. 6,258,856, U.S. Pat. No. 6,319,914, U.S. Pat. No. 6,326,365, U.S. Pat. No. 6,333,317, U.S. Pat. No. 6,334,998, U.S. Pat. No. 6,350739, U.S. Pat. No. 6,420,353, U.S. Pat. No. 6,511,969, U.S. Pat. No. 6,692,763, U.S. Pat. No. 6,844,456, U.S. Pat. No. 5,521,168; U.S. Patent Application Publication Nos. 2004/0067923, U.S. 2004/0043410, U.S. 2003/0105167, U.S. 2003/0186954, U.S. 2003/0176409, U.S. 2003/0130303, U.S. 2003/0050295, U.S. 2003/0049838, U.S. 2002/0183299, U.S. 2002/0165213, U.S. 2002/0028793, U.S. 2002/0022593, U.S. 2001/0051602; International Patent Application Publication No. WO 03/072109, WO 03/072110, WO 03/015704; European Patent No. 753,300; see also Dykens et al. (2003) Exp. Gerontol. 38(1-2):101-107; Wang et al. (2003) Invest. Ophthalmol. Vis. Sci. 44(5):2067-75; Garcia-Segura et al. (2001) Prog. Neurobiol. 63(1):29-60; Deshpande et al. (2000) Ind. J. Physiol. Pharmacol. 44(1)43-49; Behl et al. (1995) Biochem. Biophys. Res. Commun. 216:473-82; McCullough et al. (2003) Trends Endocrinol. Metab. 14(5):228-235; Kulkarni et al. (2002) Arch. Women Ment. Health 5:99-104; Zemlyak et al., 2002 Brain Res. 958:272-76; Kompoliti (2003) Front. Biosci. 8:391-400; Mooradian (1993) J. Steroid Biochem. Molec. Biol. 45(6):509-511; Kupina et al. (2003) Exp. Neurol. 180:55-73).

Estrogen does appear to play a role in the regulation of cellular gene expression (Shapiro et al., Recent Progress in Hormone Res. 45:29, 1989) and can have an effect on viral replication. For example, Almog et al. (Antiviral Res. 19:285, 1992) found, in a model of nude mice transplanted with Hep G-2 cells that contain replicating HBV, that estrogen treatment suppressed HBV DNA expression in males, but had only a minor effect on females. In contrast, Rosenbaum et al. (J. Gen. Virol. 70:2227, 1989) found that specific RNA transcripts of human papillomavirus type 16 in Siha cervical carcinoma cells are stimulated by estrogen.

### BRIEF SUMMARY

Briefly, the instant disclosure is generally directed to polycyclic phenolic compounds (PPCs) that have activity as anti-infectives, as well as to methods for their use and to pharmaceutical compositions thereof. More specifically, the compounds of this invention have the following general structure (I): 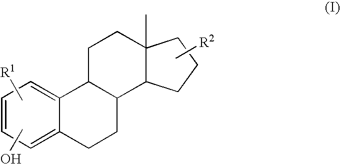
including stereoisomers, prodrugs and pharmaceutically acceptable salts or conjugates thereof, wherein R¹ and R² are as defined herein.

The compounds of this invention have utility over a wide range of therapeutic applications, and may be used to treat infectious diseases and related conditions and, in particular, viral infections. For example, certain embodiments relate to a method for treating or preventing a viral infection, comprising administering a therapeutically effective amount of a compound of structure (I) to a subject in need thereof. In certain embodiments, PPCs are useful for treating or preventing viral infections caused by Flaviviridae, Hepadnaviridae, Herpesviridae, Papillomaviridae, Retroviridae, Adenoviridae, or respiratory viruses (such as Adenoviridae, Orthomyxoviridae, Paramyxoviridae and Coronaviridae).

### BRIEF DESCRIPTION OF THE DRAWINGS

FIG. 1 shows the percentage reduction in non-cytopathic BVDV PE515 genomic RNA following a three day treatment of MDBK cells with various concentrations of compound 20 (MOI=0.001).

FIGS. 2A and 2B show the 3-dimensional and 2-dimensional, respectively, synergy plots of interferon-α2b (IFN-α) and Compound 20 against MDBK cells that were infected with BVDV at a multiplicity of infection (MOI) of 0.01. The positive % above the horizontal plane (additivity surface) reveals the regions and corresponding drug concentrations at which a synergistic effect is observed. The gradation in gray, a shift from light to dark, indicates the level of synergy.

FIG. 3 shows an isobologram of the data shown in FIG. 2.

FIGS. 4A and 4B show the 3-dimensional and 2-dimensional, respectively, cytotoxicity assay plots in which MDBK cells were infected with BVDV at a multiplicity of infection (MOI) of 0.01 and then exposed to interferon-α2b (IFN-α) and Compound 20. The negative % below the horizontal plane (additivity surface) reveal the regions and corresponding drug concentrations at which cytotoxicity is not increased and even possibly diminished. The gradation in gray, a shift from dark to light, indicates the level of antagonism (i.e., less than expected cytotoxicity).

FIG. 5 shows a plot of the fraction of virus affected against the Combination Index (Fa-CI plot) to examine the drug interaction of compound 2 and NM107 (1:5). This Monte Carlo analysis provides a measure of statistical significance because the Fa-CI plot has three lines, which represent the median value (middle line) and ±1.96 standard deviations (upper and lower lines).

FIG. 6 shows an isobologram of the experimental EC₅₀, EC₇₅, and EC₉₀ of compound 2 and NM 107 compared to their additivity line. Values to the left of their respective additivity lines indicate synergy.

### DETAILED DESCRIPTION

As set forth above, the present disclosure provides polycyclic phenolic compounds (PPCs) and compositions thereof for use in treating or preventing infectious diseases, such as those resulting from viral infections. In particular, these PPCs are useful for treating or preventing viral infections caused by Flaviviridae, Hepadnaviridae, Herpesviridae, Papillomaviridae, Retroviridae, Adenoviridae, or respiratory viruses (such as Adenoviridae, Orthomyxoviridae, Paramyxoviridae and Coronaviridae). The invention, therefore, relates generally to the surprising discovery that certain PPCs, such as tetracyclic steroid-like compounds, have antiviral activity. Accordingly, the compounds of the instant invention are useful as potential therapeutics for the prevention or treatment of viral infections and related conditions. Discussed in more detail below are PPCs suitable for use within the present invention, as well as representative compositions and therapeutic uses.

Prior to setting forth the invention in more detail, it may be helpful to an understanding thereof to set forth definitions of certain terms to be used hereinafter.

The use of the alternative (e.g., “or”) should be understood to mean either one, both, or any combination thereof of the alternatives. As used herein, the indefinite articles “a” or “an” should be understood to refer to “one or more” of any recited or enumerated component.

In the present description, any concentration range, percentage range, or integer range is to be understood to include the value of any integer within the recited range and, when appropriate, fractions thereof (such as one tenth and one hundredth of an integer), unless otherwise indicated. As used herein, “about” or “comprising essentially of” mean within an acceptable error range for the particular value as determined by one of ordinary skill in the art, which will depend in part on how the value is measured or determined, i.e., the limitations of the measurement system. For example, “about” or “comprising essentially of” can mean within 1 or more than 1 standard deviation as used in the relevant art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

As used herein, the term “alkyl” refers to a saturated or unsaturated, branched, straight-chain or cyclic monovalent hydrocarbon group derived by the removal of one hydrogen atom from a single carbon atom of a parent alkane, alkene or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, or the like. The alkyls may have any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. When a specific level of saturation is intended, the expressions “alkanyl,” “alkenyl,” and “alkynyl” are used. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Representative alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, or the like.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, or the like. The alkenyl group may be substituted or unsubstituted.

“Alkynyl” refers to an unsaturated branched, straight chain or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, or the like.

“Heteroalkyl, Heteroalkanyl, and Heteroalkenyl” refer to alkyl, alkanyl, and alkenyl groups, respectively, in which one or more of the carbon atoms (and any associated hydrogen atoms) are each independently replaced with the same or different heteroatoms or heteroatomic groups. Representative heteroatoms or heteroatomic groups that can be included in these groups include —O—, —S—, —Se—, —O—O—, —S—S—, —O—S—, —O—S—O—, —O—NR′—, —NR′—, —NR′—NR′—, ═N—N═, —N═N—, —N═N—NR′—, —PH—, —P(O)₂—, —O—P(O)₂—, —SH₂—, —S(O)₂—, —SnH₂—, or the like, and combinations thereof, including —NR′—S(O)₂—, wherein each R¹ is independently selected from hydrogen, alkyl, alkanyl, alkenyl, alkynyl, aryl, arylalkyl, heteroaryl and heteroarylalkyl, as defined herein.

“Aryl” refers to a monovalent aromatic hydrocarbon group derived by the removal of one hydrogen atom from a single carbon atom of a parent aromatic ring system. Representative aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, or the like. In certain embodiments, the aryl group is (C₅-C₁₄) aryl, with (C₅-C₁₀) being even more preferred. In other embodiments, aryls are cyclopentadienyl, phenyl and naphthyl. The aryl group may be substituted or unsubstituted.

“Arylalkyl” refers to an acyclic alkyl group in which one of the hydrogen atoms bonded to a carbon atom, typically a terminal or Sp³ carbon atom, is replaced with an aryl group. Representative arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl or the like. Where specific alkyl moieties are intended, the nomenclature arylalkanyl, arylakenyl or arylalkynyl is used. In certain embodiments, the arylalkyl group is (C₆-C₂₀) arylalkyl, e.g., the alkanyl, alkenyl or alkynyl moiety of the arylalkyl group is (C₁-C₆) and the aryl moiety is (C₅-C₁₄). In further embodiments, the arylalkyl group is (C₆-C₁₃), e.g., the alkanyl, alkenyl or alkynyl moiety of the arylalkyl group is (C₁-C₃) and the aryl moiety is (C₅-C₁₀). As used herein, “arylalkyl” substituent may be attached to a core structure (e.g., tetracyclic steroid-like compound) via the aryl moiety or via the alkyl moiety—thus, “arylalkyl” and “alkylaryl” are used interchangeably.

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Representative heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, or the like. In certain embodiments, the heteroaryl group is a 5-14 membered heteroaryl or a 5-10 membered heteroaryl. In further embodiments, heteroaryl groups are those derived from thiophene, pyrrole, benzothiophene, benzofuran, indole, pyridine, quinoline, imidazole, oxazole or pyrazine. The heteroaryl group may be substituted or unsubstituted.

“Carbocyclic” refers to a monocyclic or polycyclic compound that is saturated, unsaturated, or aromatic and is comprised of only carbon atoms, which can be optionally substituted. Exemplary carbocycles include cyclopropanyl, cyclohexanyl, pinanyl, adamantyl, 2-camphanyl, or the like.

“Heterocyclic” refers to a monocyclic or fused ring group having in the ring(s) one or more atoms selected from nitrogen, oxygen or sulfur. The rings may also have one or more double bonds. However, the rings do not have a completely conjugated π-electron system. The heterocyclic ring may be substituted or unsubstituted. When substituted, one or more substituted groups are independently selected from alkyl, aryl, haloalkyl, halo, hydroxy, alkoxy, mercapto, cyano, sulfonamidyl, aminosulfonyl, acyl, acyloxy, nitro, or substituted amino.

“Heteroarylalkyl” refers to an acyclic alkyl group in which one of the hydrogen atoms bonded to a carbon atom, typically a terminal or sp³ carbon atom, is replaced with a heteroaryl group. When one or more specific alkyl moiety is intended, the nomenclature heteroarylalkanyl, heteroarylakenyl or heterorylalkynyl is used. In certain embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In other embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

The various naphthalenecarbonyl, pyridinecarbonyl, thiophenecarbonyl and furancarbonyl groups referred to herein include the various position isomers and these can be naphthalene-1-carbonyl, naphthalene-2-carbonyl, nicotinoyl, isonicotinoyl, N-methyl-dihydro-pyridine-3-carbonyl, thiophene-2-carbonyl, thiophene-3-carbonyl, furan-2-carbonyl and furan-3-carbonyl. The naphthalene, pyridine, thiophene and furan groups can be optionally further substituted as indicated herein.

“Halogen” or “halo” refers to fluoro (F), chloro (Cl), bromo (Br), iodo (I). As used herein, —X refers to independently any halogen.

Sulphur (S) atom may be present in several compounds of this disclosure, and when present, the S atom can be at any oxidation state (e.g., S, SO, SO₂).

“Acyl” group refers to the C(O)—R″ group, where R″ is selected from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heterocyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters can include amino acid ester derivatives. The acyl group may be attached to a compound's backbone at either end of the acyl group, i.e., via the C or the R″. When an acyl group is attached via the R″, then C will bear another substituent, such as hydrogen, alkyl, heteroaryl or the like.

“Substituted” refers to a group in which one or more hydrogen atoms are each independently replaced with the same or different substituent(s). Typical substituents include —X, —R¹³, —O—, ═O, —OR, —SR¹³, —S—, ═S, —NR¹³R¹³, ═NR¹³, CX₃, —CF₃, —CN, —OCN, —SCN, —NO, NO₂, ═N₂, —N₃, —S(O)₂O—, —S(O)₂OH, —S(O)₂R¹³, —OS(O)₂O—, —OS(O)₂OH, —OS(O)₂R¹³, —P(O)(O⁻)₂, —P(O)(OH)(O⁻), —OP(O)2(O⁻), —C(O)R ³, —C(S)R¹³, —C(O)OR¹³, —C(O)O⁻, —C(S)OR¹³, —C(O)NR¹³R¹³, —C(S)NR¹³R¹³, and —C(NR³)NR¹³R¹³, wherein each X is independently a halogen; each R¹³ is independently hydrogen, halogen, alkyl, aryl, arylalkyl, arylaryl, arylheteroalkyl, heteroaryl, heteroarylalkyl NR¹⁴R¹⁴, —C(O)R¹⁴, and —S(O)₂R¹⁴; and each R¹⁴ is independently hydrogen, alkyl, alkenyl, alkynyl, aryl, arylalkyl, arylheteroalkyl, arylaryl, heteroaryl or heteroarylalkyl.

“Prodrug” herein refers to a compound that is converted into the parent compound in vivo or in a biological system. Prodrugs often are useful because, in some situations, they may be easier to administer than the parent compound. For example, the prodrug may be more bioavailable by oral administration or for cellular uptake than a parent compound. Another example is that the prodrug may have improved solubility in pharmaceutical compositions over the parent compound. A representative prodrug would be a compound of the embodiments of the present invention that is administered, for example, as an ester, phosphate, or sulfate (the “prodrug”) to facilitate transmittal across a cell membrane when water solubility is detrimental to mobility, which then is metabolically hydrolyzed to an active entity once inside the cell where water solubility is beneficial. Such a compound is generally inactive (or less active) until converted to the active form.

“Pharmaceutically acceptable salt” refers to a salt of a compound of the invention that is pharmaceutically acceptable and that possesses the desired pharmacological (e.g., antiviral) activity. Such salts include the following: (1) acid addition salts, formed with inorganic acids such as hydrochloric acid, hydrobromic acid, sulfuric acid, nitric acid, phosphoric acid, or the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, or the like; or (2) salts formed when an acidic proton present in the parent compound is replaced, for example, by a metal ion, e.g., an alkali metal ion, an alkaline earth ion, or an aluminum ion; or coordinates with an organic base such as ethanolamine, diethanolamine, triethanolamine, N-methylglucamine, or the like; or forms a conjugate with an organic acid such as a sulfate conjugate, glucoronidate conjugate, or the like.

The term “independently” means that a substituent can be the same or different for each item identified or described.

It should be understood that the individual compounds or groups of compounds derived from the various combinations of the structures and substituents described herein are disclosed by the present application to the same extent as if each compound or group of compounds were set forth individually. Thus, selection of specific structures or specific substituents or specific combinations of substituents is within the scope of the present disclosure.

Polycyclic Phenolic Compounds (PPCS)

The instant disclosure is generally directed to PPCs that have activity as anti-infectives and to pharmaceutical compositions thereof, as well as to methods for their use. More specifically, the compounds of the instant disclosure have antiviral activity and have the following general structure (I):

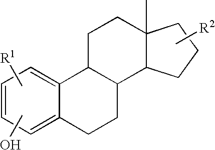

including stereoisomers, prodrugs and pharmaceutically acceptable salts or conjugates thereof, wherein:

R¹ is hydrogen, alkyl, substituted alkyl, aryl, substituted aryl, arylalkyl, substituted arylalkyl, carbocycle or substituted carbocycle;

R² is hydrogen, —OH, —O—R⁴, ═O, ═N—R³, —N(R¹⁴R¹⁴), —SH, —S—R⁴, or =S;

R³ is alkyl, substituted alkyl, aryl, substituted aryl, arylalkyl, substituted arylalkyl, carbocycle, substituted carbocycle, heterocycle, substituted heterocycle, —N(R⁴R⁵), —O—R⁴, or —N(R⁴)C(═O)R⁵;

R⁴ and R⁵ are independently selected from hydrogen, alkyl, substituted alkyl, aryl, substituted aryl, arylalkyl, substituted arylalkyl, carbocycle, substituted carbocycle, heterocycle or substituted heterocycle; and

each R¹⁴ is independently hydrogen, alkyl, alkenyl, alkynyl, aryl, arylalkyl, arylheteroalkyl, arylaryl, heteroaryl or heteroarylalkyl.

In describing the location of groups and substituents, the following numbering system will be employed, to conform the numbering of the tetracyclic steroid-like nucleus to the convention used by the IUPAC and Chemical Abstracts Service:

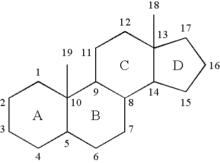

In addition, the term “steroid” is intended to mean compounds having the tetracyclic steroid-like nucleus described herein.

In certain embodiments, R² is at position 17 of the compounds of structure (I) and R² is —OH, —O—R⁴, ═O, ═N—R³, and more specifically R² is —OH(β), ═N—NH₂ or ═N—O—CH₃. In other embodiments, R¹ is at position 2 of the compounds of structure (I) and R¹ is alkyl, substituted alkyl, aryl, substituted aryl, arylalkyl, substituted arylalkyl, carbocycle or substituted carbocycle, and more specifically R¹ is a lower alkyl or substituted lower alkyl (such as isopropyl or tert-butyl or valinyl or alaninyl), or a carbocycle or substituted carbocycle (such as cyclopropanyl or cyclohexanyl or pinanyl or adamantyl or 2-camphanyl). In further embodiments, the A ring —OH is at position 3 of the compounds of structure (I). In related embodiments, R² is at position 17 and is —OH(β), —OH(α), ═N—NH₂ or ═N—O—CH₃; R¹ is at position 2 and is a lower alkyl or substituted lower alkyl (such as isopropyl or tert-butyl or valinyl or alaninyl), or a carbocycle or substituted carbocycle (such as cyclopropanyl or cyclohexanyl or pinanyl or adamantyl or 2-camphanyl); and the A ring —OH is at position 3 of the compounds of structure (I). In one embodiment, the compound of structure (I) is 17(α)-estradiol or the sodium salt of its sulfate conjugate (17(α)-estradiol-3-sulfate sodium, Compound 16). In another embodiment, the compound of structure (I) is 17(β)-2-(1-adamantyl)-estra-1,3,5(10)-triene-3,17-diol (Compound 20) or 17(β)-2-(tert-butyl)-estra-1,3,5(10)-triene-3,17-diol (Compound 21). In further embodiments, the compound of structure (I) is Compound 2, 3, 4 or 5.

In one embodiment, wherein R¹ is at position 2 and R² is at position 17, the compounds have a structure of formula (I-a):

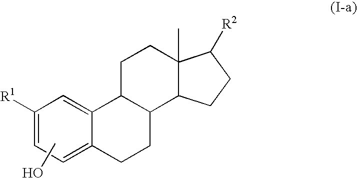

including stereoisomers, prodrugs and pharmaceutically acceptable salts or conjugates thereof, wherein:

R¹ is hydrogen, alkyl, substituted alkyl, aryl, substituted aryl, arylalkyl, substituted arylalkyl, carbocycle or substituted carbocycle;

R² is —O—R⁴, ═O, —S—R⁴, or ═S; and

R⁴ is hydrogen, alkyl, substituted alkyl, aryl, substituted aryl, arylalkyl, substituted arylalkyl, carbocycle, substituted carbocycle, heterocycle or substituted heterocycle.

In certain embodiments, R¹ is a lower alkyl or substituted lower alkyl (such as isopropyl or tert-butyl or valinyl or alaninyl) or a carbocycle or substituted carbocycle (such as cyclopropanyl or cyclohexanyl or pinanyl or adamantyl or 2-camphanyl); R² is —OH(β) or —OA(α) or ═O; and the A ring —OH is at position 3. In certain embodiments, In further embodiments, R¹ is tert-butyl or adamantyl; R² is —OA(β) or —OA(α); and the A ring —OH is at position 3.

In another embodiment, wherein R¹ is at position 2 and R² is at position 17 and is ═N—R³, the compounds have a structure of formula (I-b):

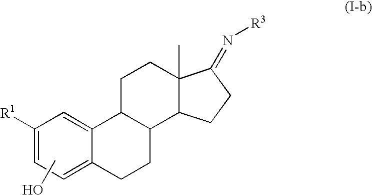

including stereoisomers, prodrugs and pharmaceutically acceptable salts or conjugates thereof, wherein:

R¹ is hydrogen, alkyl, substituted alkyl, aryl, substituted aryl, arylalkyl, substituted arylalkyl, carbocycle or substituted carbocycle;

R³ is alkyl, substituted alkyl, aryl, substituted aryl, arylalkyl, substituted arylalkyl, carbocycle, substituted carbocycle, heterocycle, substituted heterocycle, —N(R⁴R⁵), —O—R⁴, or —N(R⁴)C(═O)R⁵; and

R⁴ and R⁵ are independently selected from hydrogen, alkyl, substituted alkyl, aryl, substituted aryl, arylalkyl, substituted arylalkyl, carbocycle, substituted carbocycle, heterocycle or substituted heterocycle.

In certain embodiments, R¹ is a lower alkyl or substituted lower alkyl (such as isopropyl or tert-butyl or valinyl or alaninyl) or a carbocycle or substituted carbocycle (such as cyclopropanyl or cyclohexanyl or pinanyl or adamantyl or 2-camphanyl); ═N—R³ at position 17 is ═N—N(R^{3a}R^{3b}) or ═N—O—R^{3a} or ═N—N(R^{3a})(C═O)R^{3b}, wherein R^{3a} and R^{3b} are independently selected from hydrogen or R³; and the A ring —OH is at position 3. In further embodiments, R¹ is tert-butyl or adamantyl; ═N—R³ at position 17 is ═N—NH₂ or ═N—O—CH₃; and the A ring —OH is at position 3.

As used herein, reference to the “compounds of structure (I)” is intended to encompass all structural variants, including compounds having a structure of formula (I), (I-a), (I-b), or any combination thereof.

In certain embodiments, R¹ is tert-butyl or adamantyl and the compounds have the following structures:

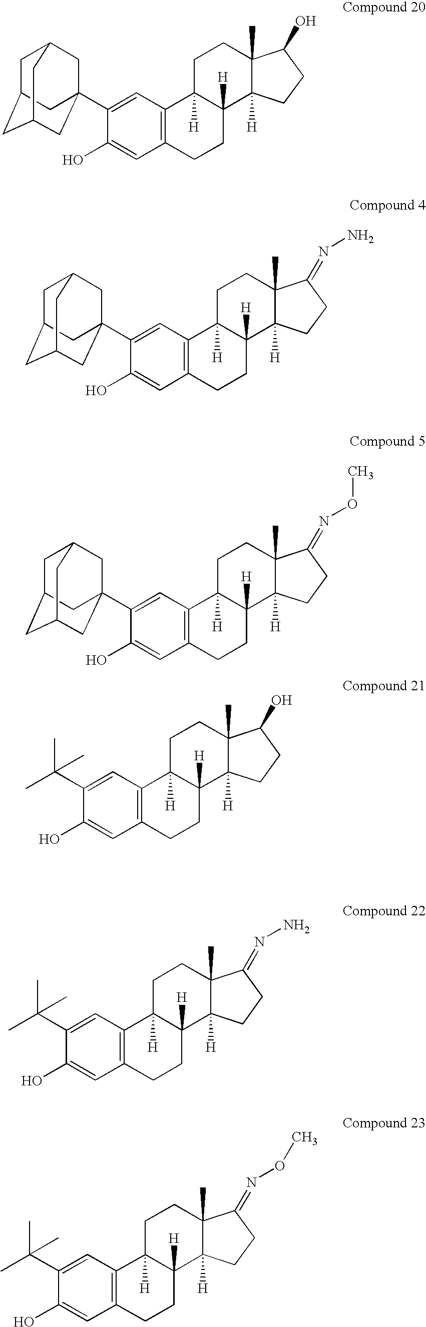

In still another embodiment, R¹ is hydrogen and the compound has the following structure:

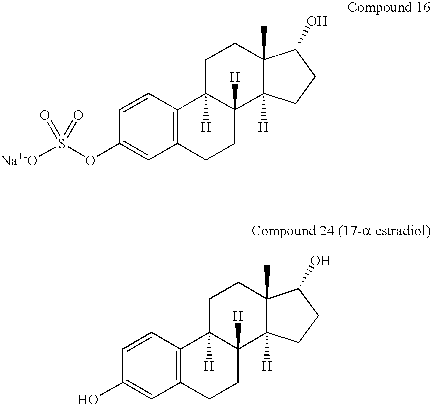

“Structurally pure” refers to a compound composition in which a substantial percentage, e.g., on the order of 95% to 100% and preferably ranging from about 95%, 96%, 97%, 98%, 99% or more, of the individual molecules comprising the composition each contain the same number and types of atoms attached to each other in the same order and with the same bonds. As used herein, “structurally pure” is not intended to distinguish different geometric isomers or different optical isomers from one another. For example, as used herein a mixture of cis-and trans-but-2,3-ene is considered structurally pure, as is a racemic mixture. When compositions are intended to include a substantial percentage of a single geometric isomer and/or optical isomer, the nomenclature “geometrically pure” and “optically or enantiomerically pure,” respectively, are used.

The phrase “structurally pure” is also not intended to discriminate between different tautomeric forms or ionization states of a molecule, or other forms of a molecule that result as a consequence of equilibrium phenomena or other reversible interconversions. Thus, a composition of, for example, an organic acid is structurally pure even though some of the carboxyl groups may be in a protonated state (—CO₂H) and others may be in a deprotonated state (—CO₂⁻). Likewise, a composition comprising a mixture of keto and enol tantomers, unless specifically noted otherwise, is considered structurally pure.

The antiviral compounds of this disclosure may contain a chiral center on any of the substituents and these can exist in the form of two optical isomers (the (+) and (−) isomers, also referred to as the (R) and (S) isomers). All such enantiomers and mixtures thereof, including racemic mixtures, are included within the scope of this disclosure. A single optical isomer (or enantiomer) can be obtained by methods known in the art, such as by chiral HPLC or other chiral chromatography, enzymatic resolutions, use of chiral auxiliaries, selective crystallization, or any combination thereof. In certain embodiments, some of the crystalline forms of the antiviral compounds of this disclosure may exist as polymorphs, which are included within the scope of this disclosure. In further embodiments, some of the antiviral compounds of this disclosure may form solvates with solvents (e.g., water, organic solvents), which are included within the scope of this disclosure.

In certain embodiments, the present disclosure provides compounds in the form of a single enantiomer that is at least 90%, 95%, 97% or at least 99% free of a corresponding enantiomer. In one embodiment, the single enantiomer is in the (+) form and is at least 90%, at least 95%, at least 97% or at least 99% free of a corresponding (−) enantiomer. In one embodiment, the single enantiomer is in the (−) form and is at least 90%, at least 95%, at least 97% or at least 99%, free of a corresponding (+) enantiomer.

The compounds of structure (I), as well as the more specific embodiments discussed herein or known in the art, may be made by techniques knows to those skilled in the field of organic chemistry, and as more specifically described in the Examples and as disclosed in U.S. Pat. Nos. 5,554,601; 5,843,934; 5,972,923; 6,319,914; 6,350,739; 6,844,456 (Compound 20) and U.S. Patent Publication No. 2003/0105167.

PPC Compositions and Combination Therapies

The present disclosure provides PPCs and compositions thereof. In addition, the present disclosure provides methods for using such compounds or compositions in treating or preventing viral infections. The treatment or prevention of viral infections may be accomplished by administering a therapeutically effective amount of a PPC having any of the structural forms described herein, or a composition thereof, such that a viral infection is treated or prevented.

Pharmaceutically acceptable carriers, diluents or excipients for therapeutic use are well known in the pharmaceutical art, and are described herein and, for example, in Remington's Pharmaceutical Sciences, Mack Publishing Co. (A. R. Gennaro, ed., 18th Edition, 1990) and in CRC Handbook of Food, Drug, and Cosmetic Excipients, CRC Press LLC (S. C. Smolinski, ed., 1992). In certain embodiments, antiviral compounds of structure (I) may be formulated with a pharmaceutically or physiologically acceptable carrier, diluent or excipient that is aqueous, such as water or a mannitol solution (e.g., about 1% to about 20% mannitol), hydrophobic carrier (e.g., oil or lipid), or a combination thereof (e.g., oil and water emulsions). In certain embodiments, any of the pharmaceutical compositions described herein are sterile. For example, sterile saline and phosphate-buffered saline at physiological pH may be used. Preservatives, stabilizers, dyes and even flavoring agents may be optionally provided in a pharmaceutical composition. For example, sodium benzoate, sorbic acid and esters of p-hydroxybenzoic acid may be added as preservatives. Id. In addition, antioxidants and suspending agents may be optionally used. Id.

Pharmaceutical compositions comprising antiviral PPCs may be manufactured by means of conventional mixing, dissolving, granulating, dragee making, levigating, emulsifying, encapsulating, entrapping or lyophilizing processes. Pharmaceutical compositions may be formulated in conventional manner using one or more physiologically acceptable carriers, diluents, excipients or auxiliaries that facilitate formulating active antiviral compounds of structure (I) into preparations that can be used pharmaceutically. A single antiviral compound of structure (I), a plurality of antiviral compounds of structure (I), or antiviral compounds of structure (I) in combination with one or more biologically active agents (e.g., other antivirals, antibacterials, antifungals, etc.) may be formulated with a pharmaceutically acceptable carrier, diluent or excipient to generate pharmaceutical compositions of the instant disclosure.

In certain embodiments, the combination therapies may be conveniently formulated together or separately in pharmaceutical formulations comprising a combination as defined above together with a pharmaceutically acceptable carrier or carriers. In further embodiments, the individual components of the noted combination therapies may be administered either concurrently or sequentially, either in separate or combined pharmaceutical formulations, each in similar or different dosage forms, each by similar or different dosage schedules, or each by the same or different routes of administration, or any combination thereof (in any order or combination), as appropriately determined by those of skill in the art.

In certain embodiments, an antiviral compound of structure (I) may be used in combination with one or more other adjunctive therapies, such as other antiviral treatments. In one aspect of the instant disclosure, the antiviral compounds of structure (I) may be utilized with one or more of a helicase inhibitor, a protease inhibitor, an α-glucosidase inhibitor, an inhibitor of an internal ribosome entry site (IRES), a compound that alters viral replication such as a polymerase inhibitor or a nucleoside analog (e.g., ribavirin, 2′-C-methyl cytidine, valopicitabine, lamivudine, zidovudine, abacavir, or derivatives thereof), a compound that alters activity of any other structural or non-structural viral protein, or a compound that alters host immune function such as thymosin-α or interferon (including α-interferon, β-interferon, γ-interferon, and derivatives thereof).

Exemplary glucosidase inhibitors for use in combination with PPCs include castanospermine and derivatives thereof (e.g., esters of castanospermine, such as celgosivir ([1S-(1α,6β,7α,8β,8αβ)]-octahydro-1,6,7,8-indolizinetetrol 6-butanoate); see, e.g., WO 01/54692; WO 02/089780). Other glucosidase inhibitors include miglitol, imino sugars such as deoxygalactonojirimycin (DGJ) or deoxynojirimycin (DNJ) or derivatives thereof (e.g., N-butyl-DNJ, N-nonyl-DNJ; see, e.g., WO 99/29321), and long alkyl chain imino sugars such as N7-oxanonyl-DNJ, N7-oxanonyl-DGJ. Each of these adjunctive therapeutics are inhibitors of ER α-glucosidases that potently inhibit the early stages of glycoprotein processing (see, e.g., Ruprecht et al., J. Acquir. Immune Defic. Syndr. 2:149, 1989; see also, e.g., Whitby et al., Antiviral Chem. Chemother. 15:141, 2004; Branza-Nichita et al., J. Virol. 75:3527, 2001; Courageot et al., J. Virol. 75:564, 2000; Choukhi et al., J. Virol. 72:3851, 1998; WO 99/29321; WO 02/089780).

Another exemplary adjunctive agent or compound for use in combination with PPCs is one that inhibits the binding to or infection of cells by a virus, such as Flaviviridae, Hepadnaviridae, Herpesviridae, Papillomaviridae, Retroviridae, Adenoviridae, or respiratory viruses (such as Adenoviridae, Orthomyxoviridae, Paramyxoviridae and Coronaviridae). Such compounds include antibodies, glucosaminoglycans (such as heparan sulfate and suramin), or the like. In certain embodiments, an antibody may be a monoclonal or polyclonal antibody, or antigen binding fragments thereof, including genetically engineered chimeric, humanized, sFv, or other such immunoglobulins. Specific examples of such compounds include antibodies that specifically bind to one or more HCV or HIV gene products or to a cell receptor to which HCV or HIV binds.

Another exemplary adjunctive agent or compound for use in combination with PPCs is one that inhibits the release of viral RNA from the viral capsid or inhibits the function of viral gene products, including inhibitors of the IRES, protease inhibitors, helicase inhibitors, and inhibitors of the viral polymerase/replicase (see, e.g., Olsen et al., Antimicrob. Agents Chemother. 48:3944, 2004; Stansfield et al., Bioorg. Med. Chem. Lett. 14:5085, 2004). Inhibitors of IRES include, for example, nucleotide sequence specific antisense (see, e.g., McCaffrey et al., Hepatology 38:503, 2003); small yeast RNA (see, e.g., Liang et al., World J. Gastroenterol. 9:1008, 2003); or short interfering RNA molecules (siRNA) that inhibit translation of mRNA; and cyanocobalamin (CNCbl, vitamin B12) (Takyar et al., J. Mol. Biol. 319:1, 2002). NS3 protease (helicase) inhibitors include peptides that are derived from NS3 substrates and act to block enzyme activity. Exemplary serine protease inhibitors, which have been investigated as potential HCV therapeutics, include BILN 2061 (see, e.g., Lamarre et al., Nature 426:186, 2003) (Boehringer Ingelheim (Canada) Ltd., Quebec), VX-950 (telaprevir) (Vertex Pharmaceuticals, Inc. Cambridge, Mass.), ITMN-191 (Intermune), GS9132/ACH806 (Gilead/Achillion), or SCH 503034 (Schering Plough). R7128 (Pharmasset/Roche), or R1626

Still another exemplary adjunctive agent or compound is one that alters viral replication, including inhibitors of RNA-dependent RNA polymerase, inhibitors of HCV p7 (e.g., DGJ and derivatives), glycoprotein processing inhibitors as described herein, nucleoside analogues including inhibitors of inosine monophosphate dehydrogenase (e.g., ribavirin, mycophenolic acid, VX497 (merimepodib, Vertex Pharmaceuticals)), other antiviral compounds such as amantadine, (Symmetrel®, Endo Pharamceuticals), rimantadine (Flumadine®, Forest Pharmaceuticals, Inc.), 2′-C-methyl cytidine (NM107, Idenix Pharmaceuticals), valopicitabine (NM283, the valine ester of NM107; Idenix Pharmaceuticals), R7128 (Pharmasset/Roche), or RI 626 (Roche); nucleotide reverse transcriptase (RT) inhibitors (e.g., lamivudine (3TC), zidovudine (ZDV), azidothymidine (AZT), zalcitabine, dideoxycytidine, dideoxyinosine, emitricitabine (FTC), stavudine (4dT), didanosine, tenofovir disoproxil fumarate, adefovir dipivoxil, abacavir, abacivir sulfate, or any combination thereof); non-nucleoside RT inhibitors (e.g., HCV-796 (Viropharma/Wyeth), nevirapine (NVP), efavirenz (EFV), delavirdine (DLV), or any combination thereof); fusion inhibitors (e.g., enfuvirtide); or protease inhibitors (e.g., amprenavir (APV), tipranavir (TPV), saquinavir, saquinavir mesylate (SQV), indinavir, lopinavir, ritonavir (RTV), fosamprenavir calcium (FOS-APV), atazanavir sulfate (ATV), nelfinavir mesylate (NFV), darunavir, or any combination thereof).

Antiviral compounds of structure (I) may be combined with an adjunctive agent or compound that ameliorates (preferably decreases or reduces the severity or intensity of, reduces the number of, or abrogates) the symptoms and effects of a viral infection. Exemplary compounds that modulate symptoms of viral infection include antioxidants, such as the flavonoids. The term “antioxidant” as defined herein in the claims refers to any molecule that prevents oxidation of a particular substrate by a second molecule. Representative antioxidant compounds includes thiols such as glutathione, taurine, cystein, homocysteine, and α-lipoic acid; istamine dihydrochloride; phenols such as probucol, salicylates, Trolox C, 3,4-dihydroxytoluene, 3,4-dihydroxycinnamic acid, nordihydroxyquaiarectic acid, 2″,4′-dihydroxyacetophenone, 2′,5′-dihydroxyacetophenone, 3′4′-dihydroxyacetophenone, propylgallate; spin trapping agents such as dimethyl-1-pyrroline-N-oxide, N-tert-butyl-α-phenylnitrone; aromatic amines such as promethazine, chlorpromazine, ethoxquin, allopurinol, uric acid; carotenoids such as β-carotene, α-carotene, γ-carotene, lypcopene, Caratol; flavonoids such as (+)-catechin, dihydroquercetin, hesperetin, texasin, biochanin A, kaempferol, quercetin, and 6,7-dhydroxy-4′-methoxy-isoflavanol; selenium aminosteroids such as trilazad mesylate, methyl prednisolone, suleptnate, lazaroids; and ubiquinones such as coenzyme Q2, coenzyme Q10; or the like.

Yet another adjunctive agent or compound is one that acts to alter immune function (increase or decrease in a statistically significant, clinically significant, or biologically significant manner). In certain embodiments, the altered immune function is enhanced or stimulated. In other embodiments, the enhanced or stimulated immune function or immune response may be against a Hepacivirus infection, such as an HCV infection, or against a Retroviridae infection, such as an HIV 1 or HIV2 infection, or Flaviviridae, Hepadnaviridae, Herpesviridae, Papillomaviridae, Adenoviridae, or respiratory viruses (such as Adenoviridae, Orthomyxoviridae, Paramyxoviridae and Coronaviridae). For example, a compound may stimulate a T cell response or enhance a specific immune response (e.g., thymosin-α, and interferons such as α-interferons and β-interferons), or may stimulate or enhance a humoral response. Exemplary compounds that alter an immune function include type I interferons, such as interferon-α (see, e.g., Nagata et al., Nature 287:401-408 (1980)), interferon-β (see, e.g., Tanigushi et al., Nature 285:547-49 (1980)), and interferon-ω (Adolf, J. Gen. Virol. 68:1669-76 (1987)), and type II interferons, such as interferon-γ (Belardelli, APMIS 103:161, 1995) and interferon-γ-1b (Actimmune®, InterMune). Exemplary interferon-α include interferon-α-2a (Roferon®-A; Hoffman-La Roche), interferon-α-2b (Intron A, PBL Biomedical), interferon-α-con-1 (Infergen®, InterMune), interferon-α-n3 (Alferon or Alferon N®, Interferon Sciences), albumin interferon-α (Albuferon-alpham™, Human Genome Sciences, Rockville, Md.) and Veldona (Amarillo Biosciences, Inc.). Exemplary interferon-β include interferon-β-1a (Avonex®, Biogen Idec; or Rebif®, Serono Inc.) and interferon-β-1b (Betaseron®, Berlex).

In certain embodiments, compounds of structure (I) are administered in combination with interferon or pegylated interferon (e.g., concomitantly, sequentially, same or different routes of administration, same or different dosing intervals, etc., as described herein), such as pegylated interferon-α. Interferon-α has been used in the treatment of a variety of viral infections, either as a monotherapy or as a combination therapy (see, e.g., Liang, New Engl. J. Med. 339:1549, 1998; Hulton et al., J. Acquir. Immune Defic. Syndr. 5:1084, 1992; Johnson et al., J. Infect. Dis. 161:1059, 1990). Interferon-α binds to cell surface receptors and stimulates signal transduction pathways that lead to activation of cellular enzymes (e.g., double-stranded RNA-activated protein kinase and RNase L that inhibit translation initiation and degrade viral RNA, respectively) that repress virus replication (see, e.g., Samuel, Clin. Microbiol. Rev. 14:778, 2001; Kaufmnan, Proc. Natl. Acad. Sci. USA 96:11693, 1999). In some embodiments, a polyethylene glycol moiety is linked to interferon-α (known as pegylated interferon-α; peginterferon α-2b (Peg-Intron®; Schering-Plough) and peginterferon α-2a (Pegasys®; Hoffmann-La Roche)), which have an improved pharmacokinetic profile and also manifest fewer undesirable side effects (see, e.g., Zeuzem et al., New Engl. J. Med. 343:1666, 2000; Heathcote et al., New Engl. J. Med. 343:1673, 2000; Matthews etal., Clin. Ther. 26:991, 2004).

In certain embodiments, a composition comprising a compound of structure (I) in combination with another antiviral compound (e.g., a compound that alters immune function) may act synergistically in the treatment of a viral infection, such as a Flaviviridae infection or a Retroviridae infection. Two or more compounds that act synergistically act such that the combined effect of the compounds is greater than the sum of the individual effects of each compound when administered alone (see, e.g., Berenbaum, Pharmacol. Rev. 41:93, 1989). For example, a combination of antiviral compounds of structure (I) with another agent or compound may be analyzed by a variety of mechanistic and empirical models (see, e.g., Ouzounov et al., Antivir. Res. 55:425, 2002). A commonly used approach for analyzing synergy between a combination of agents employs the construction of isoboles (iso-effect curves, also referred to as isobolograms), in which the combination of agents (dₐ,d_{b}) is represented by a point on a graph, the axes of which are the dose-axes of the individual agents (see, e.g., Ouzounov et al., supra; see also Tallarida, J. Pharmacol. Exp. Therap. 298:865, 2001).

Another method known in the art for analyzing the effect of drugs on each other (antagonism, additivity, synergism) includes determination of combination indices (CI) according to the median effect principle to provide estimates of EC₅₀ values of compounds administered alone and in combination (see, e.g., Chou, in Synergism and Antagonism Chemotherapy. Eds. Chou and Rideout. Academic Press, San Diego Calif., pages 61-102, 1991; CalcuSyn™ software). A CI value of less than one represents synergistic activity, equal to one represents additive activity, and greater than one represents antagonism.

Still another exemplary method is the independent effect method (Pritchard and Shipman, Antiviral Research 14:181, 1990; Pritchard and Shipman, Antiviral Therapy 1:9, 1996; MacSynergy™ II software, University of Michigan, Ann Arbor, Mich.). MacSynergy™ II software allows a three-dimensional (3-D) examination of compound interactions by comparing a calculated additive surface to observed data to generate differential plots that reveal regions (in the form of a volume) of statistically greater than expected (synergy) or less than expected (antagonism) compound interactions. For example, a composition comprising a compound of structure (I) and an agent that alters immune function will be considered to have synergistic activity or have a synergistic effect when the volume of synergy produced as calculated by the volume of the synergy peaks is about 15% greater than the additive effect (that is, the effect of each agent alone added together), or about a 2-fold to 10-fold greater than the additive effect, or about a 3-fold to 5-fold or more greater than the additive effect.

The formulations of the present disclosure, having an amount of one or more antiviral compounds of structure (I), with or without other adjunctive therapies, sufficient to treat or prevent a viral infection are, for example, suitable for topical (e.g., creams, ointments, skin patches, eye drops, ear drops, shampoos) application or administration. Other exemplary routes of administration include oral, parenteral, sublingual, bladder wash-out, vaginal, rectal, enteric, suppository, nasal, or inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, and intraurethral injection or infusion techniques.

Another exemplary route of administration would be through the use of an implant. For example, a DUROS® (Alza Corp.) implant is a miniature cylinder made from a titanium alloy, which protects and stabilizes a drug formulation inside, which allows water to enter into one end of the cylinder through a semipermeable membrane and the drug(s) are delivered from a port at the other end of the cylinder at a controlled rate appropriate to the specific therapeutic agent. The pharmaceutical compositions, or devises containing such compositions, of the present disclosure are formulated so as to allow the antiviral compounds of structure (I) contained therein to be bioavailable upon administration of the composition to a subject. The level of antiviral compound in serum and other tissues after administration or implantation can be monitored by various well-established techniques, such as chromatographic-based assays. In certain embodiments, antiviral compounds of structure (I) are formulated for topical application to a target site on a subject in need thereof, such as an animal or a human. In other embodiments, antiviral PPCs are formulated for parenteral administration to a subject in need thereof (e.g., having a viral infection), such as an animal or a human.

Proper formulation is generally dependent upon the route of administration chosen, as is known in the art. For example, in exemplary embodiments for topical administration, the antiviral compounds of structure (I) may be formulated as solutions, gels, ointments, creams, suspensions, pastes, and the like. Topical formulations may contain a concentration of the compound of from about 0.1 to about 10% w/v (weight per unit volume). Systemic formulations are another embodiment, which includes those designed for administration by injection, e.g. subcutaneous, intravenous, intramuscular, intrathecal or intraperitoneal injection, as well as those designed for transdermal, transmucosal, oral, intranasal, or pulmonary administration. In one embodiment, the systemic formulation is sterile. In embodiments for injection, the antiviral compounds of structure (I) may be formulated in aqueous solutions, preferably in physiologically compatible solutions or buffers such as Hanks's solution, Ringer's solution, mannitol solutions or physiological saline buffer. In certain embodiments, any of the compositions described herein may optionally contain formulatory agents, such as suspending, stabilizing or dispersing agents. Representative compositions and preparations may be prepared as a parenteral/systemic dosage unit contains between 0.01 to 1% by weight of a compound.

Alternatively, the antiviral compounds of structure (I) may be in solid (e.g., powder) form for constitution with a suitable vehicle (e.g., sterile pyrogen-free water) before use. In embodiments for transmucosal administration, penetrants, solubilizers or emollients appropriate to the barrier to be permeated may be used in the formulation. For example, 1-dodecylhexahydro-2H-azepin-2-one (Azone®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

In other embodiments, the antiviral compounds of structure (I) can be formulated with a pharmaceutically acceptable carrier in the form of tablets, pills, dragees, capsules, liquids, gels, syrups, slurries, suspensions and the like, for oral ingestion by a subject or patient to be treated. In certain embodiments for oral solid formulations, such as powders, capsules or tablets, suitable excipients include fillers, such as sugars (e.g., lactose, sucrose, mannitol, sorbitol); cellulose preparations such as maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP); granulating agents; or binding agents. Optionally, disintegrating agents may be added, such as cross-linked polyvinylpyrrolidone, agar, or alginic acid (or a salt thereof, such as sodium alginate). If desired, solid dosage forms may be sugar-coated or enteric-coated using standard techniques. In some embodiments for oral liquid preparations, such as suspensions, elixirs or solutions, suitable carriers, excipients or diluents include water, glycols, oils, alcohols, or combinations thereof. Additionally, flavoring agents, preservatives, viscosity-increasing agents, humectants, coloring agents, or the like, may be added. In embodiments for oral or buccal administration, the compositions may take the form of, for example, tablets or lozenges, formulated as is known in the art and described herein.

In embodiments for administration by inhalation, the compounds for use according to the present disclosure may be formulated for convenient delivery in the form of drops for intranasal administration, or in the form of an aerosol spray from pressurized packs or nebulizer having a suitable propellant (e.g., dichlorodifluoromethane, trichlorofluoromethane, dichlorotetrafluoroethane, carbon dioxide or other suitable gas). In certain embodiments, the drops or aerosol composition is sterile. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base, such as lactose or starch.

In other embodiments, the antiviral compounds of structure (I) may be formulated into rectal or vaginal compositions such as suppositories or retention enemas, e.g., containing conventional suppository bases, such as lanolin, cocoa butter, polyethylene glycol or other glycerides. In the methods of the invention, the compound of structure (I) as described herein may be administered through use of an insert, bead, timed-release formulation, patch or fast-release formulation.

In addition to the formulations described herein, the antiviral compounds may also be formulated as a depot preparation. For example, antiviral compounds of structure (I) can be in the form of the slow-release formulation such that they can provide activity over time. Such long-acting formulations may be administered by implantation (for example, subcutaneously or intramuscularly) or by intramuscular injection. In certain embodiments, the compounds may be formulated with suitable a polymer (including poly(lactides), poly(glycolides), poly(caprolactones), and blends thereof), a hydrophobic material, (including a physiologically acceptable oil, which can be in the form of an emulsion), an ion exchange resin, or as sparingly soluble derivatives (such as a sparingly soluble salt).

Alternatively, other pharmaceutical delivery systems may be employed. In certain embodiments, the compounds are formulated with liposomes or emulsions as delivery vehicles. Certain organic solvents, such as dimethylsulfoxide (DMSO), may also be employed. Additionally, the antiviral compounds of structure (I) may be delivered using a sustained-release system, such as semipermeable matrices of solid or semi-solid polymers (e.g., thermopaste) containing the therapeutic agent. Sustained-release capsules may, depending on their chemical nature, release the compounds for a few hours, a few days, a few weeks, or for up to about 100 days. In still further embodiments, the antiviral compounds of structure (I) may be administered by electrically-assisted delivery (e.g., electroporation).

As certain of the carboxyl groups of the antiviral compounds of structure (I) are acidic, or the substituents R¹, R², R³, R⁴, and R⁵ may include acidic or basic substituents, the antiviral compounds of structure (I) may be included in any of the above-described formulations as a free acid, a free base, or as a pharmaceutically acceptable salt or conjugate thereof. Pharmaceutically acceptable salts and conjugates are those salts or conjugates that substantially retain the antiviral activity of the free acid or base, and which are prepared by reaction with a base or acid, respectively. Suitable acids and bases are well known to those of ordinary skill in the art and are described herein. Exemplary pharmaceutical salts may tend to be more soluble in aqueous and other protic solvents than is the corresponding free base or acid form.

Antiviral compounds of structure (I) can be provided in dosage amounts and intervals, which can be adjusted on a case-by-case basis as described herein, to provide plasma levels of one or more of the antiviral compounds sufficient to maintain a therapeutic effect. Exemplary clinical dosages for administration by injection may range from about 0.1 to about 200 mg/kg/day, or range from about 1.5 to about 15 mg/kg/day. The use of the minimum dosage sufficient to provide effective therapy is generally preferable. In certain embodiments, therapeutically effective serum levels may be achieved by administering a single dose or as a single daily dose or multiple doses each day over a specified time period. That is, the desired dose may be conveniently provided in divided doses administered at appropriate intervals, for example, two, three, four or more doses per day, or one dose per day, one dose per two days, etc. In other embodiments, therapeutically effective serum levels may also be achieved by administering at less frequent dosing schedules such as, for example, once every two days, twice a week, once a week or at longer intervals between dosing, or any combination thereof. For example, combination administration schedules may be utilized to reach therapeutically effective does, such as multiple doses on one or more days followed by less frequent dosing such as, for example, once every two days, twice a week or once a week, or longer. Patients may generally be monitored for therapeutic or prophylactic effectiveness using assays suitable for the viral infection or associated condition being treated or prevented, which will be familiar to those having ordinary skill in the art.

The antiviral compositions of this disclosure may be administered to a subject as a single dosage unit form (e.g., a tablet, capsule, injection or gel), or the compositions may be administered, as described herein, as a plurality of dosage unit forms (e.g., in aerosol or injectable form, tablet, capsule), or in any combination thereof. For example, the antiviral formulations may be sterilized and packaged in single-use, plastic laminated pouches or plastic tubes of dimensions selected to provide for routine, measured dispensing. In one example, the container may have dimensions anticipated to dispense 0.5 mL of the antiviral composition (e.g., a drop, gel or injection form) to a subject, or to a limited area of a target surface on or in a subject, to treat or prevent an infection. A target surface, for example, may be in the immediate vicinity of a skin infection or an organ (e.g., liver), where the target surface area will depend on the extent of an infection.

In cases of local administration or selective uptake, the effective local concentration of antiviral PPCs may not be related to plasma concentration. A person having ordinary skill in the art will be able to optimize therapeutically effective local dosages without undue experimentation. The amount of an active antiviral compound of structure (I) administered will be dependent upon, among other factors, the physical condition of the subject being treated, the subject's weight, the severity and longevity of the affliction being treated, the particular form of the active ingredient, the manner of administration and the composition employed, and the judgment of the prescribing physician.

Liposomal suspensions may also be pharmaceutically acceptable carriers. These may be prepared according to methods known to those skilled in the art (for example, U.S. Pat. No. 4,522,811; U.S. Pat. No. 6,320,017; U.S. Pat. No. 5,595,756). For example, liposome formulations may be prepared by dissolving appropriate lipid(s) (such as stearoyl phosphatidyl ethanolamine, stearoyl phosphatidyicholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, or triphosphate derivatives is then introduced into the container. The container is then swirled by hand to free lipid material from the sides of the container and to disperse lipid aggregates, thereby forming the liposomal suspension. Hydrophilic compounds will likely be loaded into the aqueous interior of a liposome.

The antiviral compositions may be provided in various forms, depending on the amount and number of different pharmaceutically acceptable excipients present. For example, the compositions may be in the form of a solid, a semi-solid, a liquid, a lotion, a cream, an ointment, a cement, a paste, a gel, or an aerosol. In one embodiment, the antiviral formulation is in the form of a liquid or a gel. The pharmaceutically acceptable excipients suitable for use in the antiviral formulation compositions as described herein may optionally include, for example, a viscosity-increasing agent, a buffering agent, a solvent, a humectant, a preservative, a chelating agent (e.g., EDTA or EGTA), an oleaginous compound, an emollient, an antioxidant, an adjuvant, or the like. Exemplary buffering agents suitable for use with the antiviral compounds of structure (I) or compositions thereof include monocarboxylate or dicarboxylate compounds (such as acetate, fumarate, lactate, malonate, succinate, or tartrate). Exemplary preservatives include benzoic acid, benzyl alcohol, phenoxyethanol, methylparaben, propylparaben, and the like. The function of each of these excipients is not mutually exclusive within the context of the present invention. For example, glycerin may be used as a solvent or as a humectant or as a viscosity-increasing agent.

Therapeutic Methods

The present disclosure provides methods for treating or preventing a viral infection in a host comprising administering a therapeutically effective amount of an antiviral compound of structure (I), alone or in combination with an adjunctive therapeutic agent. In one embodiment, the viral infection being treated or prevented is a Flaviviridae, Hepadnaviridae, Herpesviridae, Papillomaviridae, Retroviridae, Adenoviridae, or respiratory virus (such as Adenoviridae, Orthomyxoviridae, Paramyxoviridae and Coronaviridae) infection. The antiviral therapy may be repeated intermittently while infections are detectable or even when they are not detectable.

Treatment, as provided by the present disclosure, encompasses prophylaxis or preventative administration of any combination described herein. For example, effective treatment of a viral infection may include a cure of the infection (i.e., eradication of the virus from the host or host tissue); a sustained response in which viral RNA or DNA is no longer detectable in the blood of the subject for a certain period after completing a therapeutic regimen (such a sustained response may be equated with a favorable prognosis and may be equivalent to a cure); slowing or reducing any associated tissue damage (e.g., subjects infected with HCV may have associated liver scarring (fibrosis)); slowing or reducing production of virus; reducing, alleviating, or abrogating symptoms in a subject; or preventing symptoms or infection from worsening or progressing.

For example, if an infection is caused by or associated with HCV, the compositions described herein may be used for accomplishing at least one of the following goals: (1) elimination of infectivity and potential transmission of an HCV infection to another subject; (2) arresting the progression of liver disease and improving clinical prognosis; (3) preventing development of cirrhosis and hepatocellular carcinoma (HCC); (4) improving the clinical benefit of currently used therapeutic molecules or modalities; and (5) improving the host immune response to HCV infection. To date, a therapeutic agent that adequately treats or prevents an HCV infection and any associated disease without severe side-effects has remained elusive.

In one embodiment, the therapy or prophylaxis may be for the treatment or prevention of disease associated with an infection by Flaviviridae. The flavivirus group (family Flaviviridae) comprises the genera Flavivirus, Pestivirus and Hepacivirus. One significant member of the Hepacivirus genus is hepatitis C virus (HCV), such as genotypes 1-6 or others yet identified. HCV was first identified in 1989 and is a major cause of acute hepatitis, responsible for most cases of post-transfusion non-A, non-B hepatitis. HCV is recognized as a major cause of chronic liver disease, including cirrhosis and liver cancer (Hoofnagle, Hepatology 26:15S, 1997). The World Health Organization estimates that close to 170 million people worldwide (i.e., 3% of the world's population) are chronically infected with HCV (Global surveillance and control of hepatitis C. Report of a WHO Consultation organized in collaboration with the Viral Hepatitis Prevention Board, Antwerp, Belgium. J Viral Hepat.6:35, 1999). In the United States alone, 2.7 million people are chronically infected with HCV with an estimated 8,000 to 10,000 deaths annually (Alter et al., N. Engl J. Med. 341:556, 1999). Approximately 3-4 million people are newly infected each year, and 80-85% of these infected patients develop chronic infection with approximately 20-30% of these patients progressing to cirrhosis and end-stage liver disease, frequently complicated by hepatocellular carcinoma (HCC) (see, e.g., Kolykhalov et al., J. Virol. 74:2046, 2000).

HCV is difficult to propagate efficiently in cell culture, which renders analysis and identification of potential anti-HCV agents difficult. In the absence of a suitable cell culture system capable of supporting replication of human HCV and re-infection of cells in vitro, use of another member of the Flaviviridae family, Bovine Viral Diarrhea virus (BVDV) is an art-accepted surrogate virus for use in cell culture models (Buckwold et al., Antiviral Res. 60: 1, 2003; Stuyver et al., Antimicrob. Agents Chemother. 47:244, 2003; Whitby et al., supra). HCV and BVDV share a significant degree of local protein homology, a common replication strategy, and probably the same subcellular location for viral envelopment. Both HCV and BVDV have single-stranded genomes (approximately 9,600 and 12,600 nucleotides, respectively) that encode nine functionally analogous gene products, including the E1 and E2 envelope glycoproteins (see, e.g., Rice, Flaviviridae: The Viruses and Their Replication, in Fields Virology, 3rd Ed. Philadelphia, Lippincott, 931, 1996). Other assays well-known in the art include HCV pseudoparticles (see, e.g., Bartosch et al., J. Exp. Med. 197:633, 2003; Hsu et al., Proc. Nat'l Acad. Sci. USA 100:7271, 2003) and HCV replicons of any type, such as full length replicons, expressing E1 and E2, and also resistant to IFN-α or ribavirin (see, e.g., U.S. Pat. Nos. 5,372,928; 5,698,446; 5,874,565; 6,750,009), and full-length HCV genome that replicates and produces virus particles that are infectious in cell culture (HCVcc) assays (see, e.g., Lindenbach et al., Science 309:623, 2005).

Exemplary species of the Pestivirus genus are bovine viral diarrhea virus, Classical Swine fever virus, Border disease virus, and Hog Cholera virus. Exemplary species of the Flavivirus genus are Yellow Fever virus, Banzi virus, Bouboui virus, Edge Hill virus, Jugra virus, Saboya virus, Sepik virus, Uganda S virus, Wesselsbron virus, Entebbe virus, Yokose virus, Dengue virus, Kedougou virus, Aroa virus, Japanese encephalitis virus, Cacipacore virus, Koutango virus, Murray Valley encephalitis virus, Rocio virus, West Nile virus, Yaounde virus, Kokobera virus, Ntaya virus, Bagaza virus, Ilheus virus, Tembusu virus, St. Louis encephalitis virus, Usutu virus, Tick-Borne encephalitis virus, Louping ill virus, Powassan virus, Omsk hemorrhagic fever virus, Kyasanur Dorest disease virus, Gadgets Gully virus, Kadam virus, Langat virus, Royal Farm virus, Modoc virus, Apoi virus, Jutiapa virus, Sal Vieja virus, San Perlita virus, Rio Bravo virus, Montana myotis leukoencephalitis virus, Carey Island virus, and Cell fusing agent virus.

In certain embodiments, for example, the therapy or prophylaxis may be the treatment or prevention of a disease caused by Flaviviridae, such as hepatitis C, yellow fever, dengue fever, Japanese encephalitis, Murray Valley encephalitis, Rocio virus infection, West Nile fever, St. Louis encephalitis, tick-borne encephalitis, Louping ill virus infection, Powassan virus infection, Omsk hemorrhagic fever, Kyasanur forest disease, bovine viral diarrhea, classical swine fever, border disease, and hog cholera. A “Flaviviridae infection” or an “HCV infection” refers to any state or condition that involves (i.e., is caused, exacerbated, or characterized by) a Flaviviridae residing in the cells or body of a subject or patient. A patient or subject may be a human, a non-human mammal, sheep, cattle, horse, pig, dog, cat, rat, or mouse, or other animal.

In another embodiment, the therapy or prophylaxis may be for the treatment or prevention of disease associated with an infection by Hepadnaviridae. The Hepadnaviridae family comprises the genera Orthohepadnavirus and Avihepadnavirus. The Orthohepadnavirus genus includes Hepatitis B virus (HBV), Woodchuck Hepatitis B virus (WHBV), and Woolly Monkey Hepatitis B virus (WMHBV). The Avihepadnavirus genus includes Duck Hepatitis B virus (DHBV) and Heron Hepatitis B virus (HHBV). In certain embodiments, the therapy or prophylaxis may be the treatment or prevention of a disease caused by Hepadnaviridae, such as hepatitis B.

In still another embodiment, the therapy or prophylaxis may be for the treatment or prevention of disease associated with an infection by Herpesviridae. The Herpesviridae family comprises the genera Simplexvirus, Varicellovirus, Mardivirus, Iltovirus, Cytomegalovirus, Muromegalovirus, Roseolovirus, Lymphocryptovirus, Rhadinovirus, and Ictalurivirus. The Simplexvirus genus includes human herpes simplex virus 1 and herpes simplex virus 2; the Varicellovirus genus includes human varicella zoster virus (human herpes virus 3); the Mardivirus genus includes Marek's disease herpes virus 1 and Marek's disease herpesvirus 2; the Iltovirus genus includes Infectious laryngotracheitis virus; the Cytomegalovirus genus includes human cytomegalovirus (human herpes virus 5); the Roseolovirus genus includes human herpes simplex virus 6, 6A, 6B and 7; the Lymphocryptovirus genus includes Epstein-Barr virus (human herpes simplex virus 4); and the Rhadinovirus genus includes Woodchuck herpesvirus marmota, malignant catarrhal fever virus and Kaposi's sarcoma-associated herpes virus (human herpes simplex virus 8). In certain embodiments, the therapy or prophylaxis may be the treatment or prevention of a disease caused by Herpesviridae, such as herpes labial (oral herpes), genital herpes, herpes esophagitis, shingles, herpes stomatitis, eye disease, skin disease, or the like.

In a further embodiment, the therapy or prophylaxis may be for the treatment or prevention of disease associated with an infection by Papillomaviridae. The Papillomaviridae family comprises 16 different genera (alpha, beta, gamma, delta, epsilon, zeta, eta, theta, iota, kappa, lambda, mu, nu, xi, omikron and pi), which includes human papillomavirus (HPV) species 1 to about 96 (such as low risk HPV 6 and 11, and high risk HPV 16 and 18). In certain embodiments, the therapy or prophylaxis may be the treatment or prevention of a disease caused by Papillomaviridae, such as skin warts, gential warts (condyloma acuminate), epidermodysplasia verruciformis, respiratory papillomatosis, laryngeal papillomatosis, cervical carcinoma, or the like.

In still a further embodiment, the therapy or prophylaxis may be for the treatment or prevention of disease associated with an infection caused by a respiratory virus, such as Adenoviridae(see below), Paramyxoviridae, Coronaviridae and Orthomyxoviridae. The Paramyxoviridae include human parainfluenza virus 1-4, mumps virus, measles virus, human respiratory syncytial virus (RSV), or the like. The Coronaviridae include infectious bronchitis virus, human corona virus 229E or OC43, severe acute respiratory virus (SARS), human torovirus, or the like. The Orthomyxoviridae include influenza A virus, influenza B virus, influenza C virus, Dhori virus, Batken virus, Thogoto virus, or the like. In certain embodiments, the therapy or prophylaxis may be the treatment or prevention of a disease caused by Orthomyxoviridae, such as influenza A, B or C virus. In certain embodiments, the therapy or prophylaxis may be the treatment or prevention of a disease caused by a respiratory virus, such as RSV disease, influenza or SARS.

In yet a further embodiment, the therapy or prophylaxis may be for the treatment or prevention of disease associated with an infection caused by Retroviridae, which includes the Lentivirus genus. Exemplary Retroviridae include Rous sarcoma virus, human immunodeficiency virus (such as HIV-1 and HIV-2), HIV that are resistant to current therapy, or the like. In certain embodiments, the therapy or prophylaxis may be the treatment or prevention of a disease caused by Retroviridae, such as HIV.

In another embodiment, the therapy or prophylaxis may be for the treatment or prevention of disease associated with an infection caused by Adenoviridae, which can also be the cause of a respiratory infection. Exemplary Adenoviridae include human adenovirus A, B, C, D, E, or the like. In certain embodiments, the therapy or prophylaxis may be the treatment or prevention of a disease caused by Adenoviridae, such as human adenovirus A, B, C, D, or E.

Unless otherwise indicated, assays to detect activity of compounds of structure (I) against all of the viruses noted herein are known in the art (see, e.g., a variety of assays for testing activity on the above noted viruses at www.niaid-aacf.org/screeningassays.htm; see also Kort et al. (1993) Antimicrob. Agents Chemother. 37:115; Volsky et al. (1992) Antiviral Res. 17:335) and are incorporated herein by reference.

In certain embodiments, methods of treating or preventing any of the viral infections described herein comprise administering a compound of structure (I). In other embodiments, methods of treating or preventing any of the viral infections described herein comprise administering a composition comprising a compound of structure (I) (such as 17(β)-2-(1-adamantyl)-estra-1,3,5(10)-triene-3,17-diol or 17(β)-2-(tert-butyl)-estra-1,3,5(10)-triene-3,17-diol) and an adjunctive therapeutic agent, such as (a) a compound that inhibits viral infection of cells (including viral specific antibodies); (b) a compound that alters viral replication; (c) a helicase inhibitor; (d) a protease inhibitor (including BILN 2061, VX-950 (telaprevir), ITMN-191, GS9132/ACH806, SCH 503034, APV, TPV, SQV, indinavir, lopinavir, RTV, or any combination thereof); (e) a glucosidase inhibitor (including castanospermine or derivatives thereof, including [1S-(1α,6β,7α,8β, 8αβ)]-octahydro-1,6,7,8-indolizinetetrol 6-butanoate); (f) an inhibitor of an internal ribosome entry site (IRES); (g) a nucleoside analog (including ribavirin, 2′-C-methyl cytidine, valopicitabine, R7128, R1626, AZT, ZDV, 3TC, FTC, or any combination thereof); (h) a non-nucleoside inhibitor (such as an RT inhibitor, including NVP, EFV, DLV, HCV-796, or any combination thereof, (i) a compound that ameliorates the symptoms or effects of a viral infection (including an antioxidant); or (j) a compound that alters host immune function (including thymosin-α or interferon).

All of the U.S. patents, U.S. patent application publications, U.S. patent applications, foreign patents, foreign patent applications, non-patent publications and web-sites referred to in this specification and/or listed in the Application Data Sheet, are incorporated herein by reference, in their entirety. The invention having been described, the following examples are intended to illustrate, and not limit, the invention.

### EXAMPLES

### Example 1

### In Vitro Inhabition of Viral Release in BVDV-Infected MDBK Cells

Madin-Darby Bovine Kidney Cells (MDBK) (American Type Culture Collection (ATCC) No. CCL-22, Manassas, Va) were seeded into 96-well plates at a density of approximately 2×10⁴ cells per well. The cell cultures were incubated for 3-24 hours to allow for attachment of cells to the culture plates prior to infection and addition of compounds. BVDV strain NADL (ATCC No. VR-534) plaque forming units (PFU) were added to each well to achieve a multiplicity of infection (MOI) ranging from about 1 or about 0.001, and allowed to remain in contact with the cultured cells for 1 to 2 hours. The virus was then removed and the cells were washed with growth medium (e.g., Dulbecco's Modified Eagles Medium (DMEM)) containing 5% horse serum (HS), or with phosphate buffered saline (PBS), 1% HS, and 1 mM MgCl₂. The test compounds, dissolved in cell growth media with 2% HS, were then added to the BVDV-infected cells or uninfected cells (cytotoxicity determination) at various concentrations. The plates were incubated at 37° C., 5% CO₂ for 24 hours (i.e., about one cycle of BVDV replication). The plates were then centrifuged at low speed and the supernatant was serially diluted for use in infecting a new monolayer of cells in 12-well plates. The cell monolayer was then overlaid with 0.5% agarose dissolved in cell growth media containing 5% HS and either Compound 20; ribavirin (Sigma Co.); or IFN α (PBL Biomedical Laboratories, Piscataway, N.J.); or without a test compound (control). The treated cells were incubated for 3 to 5 days at 37° C. under 5% CO₂. The MDBK cells were fixed with formaldehyde and stained with crystal violet or methylene blue and then washed in double distilled water. Following the wash in distilled water, the plates were air dried at room temperature. The virus plaques formed in MDBK cell cultures were quantified by a manual count and a titer was determined. The data are presented in Table 1 and Table 2.

TABLE 1
Inhibition of Viral Release by Compound 20 After 24 h
Treatment Compared to Control

| Viral titer in | Viral titer in | % Inhibition of |
|---|---|---|
| untreated | presence of 1 μM of | Viral Release by |
| control (PFU/mL) | Compound 20 (PFU/mL) | Compound 20 at 1 μM |
| 7.0 × 10⁵ | 2.2 × 10⁵ | 69% |

PFU: Plaque forming units
$\begin{matrix}\% Inhibition of Viral Release = \\ 100 - ( \frac{Viral titer in presence of 1 μM}{Viral titer in untreated control} ) \times 100 \end{matrix}$

TABLE 2
Cytotoxicity of Compound 20 After 24 h Treatment
Compared to the Control

| OD₄₉₀ - | OD₄₉₀ - 1 μM | % Cytotoxicity of |
|---|---|---|
| untreated control | Compound 20 | Compound 20 (1 μM) |
| 1.2 | 1.2 | 0% |

$\% Cytotoxicity = 100 - ( \frac{{OD}_{490} in presence of 1 μM}{{OD}_{490} in untreated control} ) \times 100$

### Example 2

### Protcetion of MDBK Cells from BVDV-Induced Cytopathicity

Cell proliferation was monitored using a non-radioactive MTS/PMS assay (MTS: 3-(4,5-dimethylthiazol-2-yl)-5-(3-carboxymethoxy phenyl)-2-(4-sulfophenyl)-2H-tetrazolium (Promega, Madison, Wis.)); (PMS: phenazine methosulfate (Sigma Aldrich, St. Louis, Mo.)). MDBK cells were seeded into 96-well plates at a density of approximately 2×10⁴ cells per well. The cultures were incubated 3-24 hours to permit attachment of the cells to the plates prior to infection and addition of compounds. The appropriate PFU of BVDV-NADL were added to each well to achieve the desired MOI (<1 or >0.001); the cells were exposed to the virus diluted at the appropriate concentration in phosphate buffered saline (PBS) containing 1% horse serum (HS) for 1 to 2 hours. The virus was then removed and the cells were washed with PBS containing 1% HS. The test compounds were serially diluted in cell growth media with 2% HS and added to the cells. The plates were incubated at 37° C. in the presence of 5% CO₂ for 3-4 days. Uninfected cells and infected, untreated cells (without compound) were used as additional controls. The final volume was 100 μl per well. After the 3-4 days of incubation a volume of 20 μL of the combined MTS/PMS solution was added into each well of the 96-well assay plate containing 100 μL of cells in culture medium to obtain final concentrations of 333 μg/ml MTS 5 and 25 μM PMS. A 96-well microtiter spectrophotometer plate reader was used to measure the absorbance at 490 nm after incubation of the 96-well plate for 1 to 4 hours at 37° C. in a humidified, 5% CO₂ atmosphere. The mean absorbance in each set of triplicate wells was determined. Antiviral activity was measured as MTS conversion relative to the differential between the conversion for cell (non-infected) and viral (non-drug-treated) controls. The cytopathic effect (CPE) reduction for each concentration of the tested compound, which correlated with antiviral activity, was calculated as follows. % CPE reduction=[(D−ND)/(NI−ND)]×100, where D (drug-treated)=the absorbance of drug-treated cells; ND (non drug-treated)=the absorbance of untreated infected cells; and NI (non-infected)=the absorbance of non-infected cells. EC₅₀ represents the concentration of drug that protects 50% of the cells from BVDV induced cytotoxicity (50% CPE reduction). CC₅₀ equals the concentration that affects the viability of 50% of the MDBK cells. The data are presented in Tables 3 and 4.

TABLE 3
Protection of MDBK Cells from BVDV-Induced
Cytopathicity (MOI = 0.05)

| Compound | EC₅₀ | CC₅₀ |
|---|---|---|
| 20 | 0.80 ± 0.22 μM | 40.05 ± 25.10 |
| 4 | 3.99 ± 0.99 μM | 34.49 ± 23.16 |

TABLE 4
Multiplicity of infection (MOI) effect on the efficacy
of Compound 20 against BVDV

| | | | | |
|---|---|---|---|---|
| MOI | 0.01 | 0.02 | 0.03 | 0.05 |
| EC₅₀ (μM) | 0.34 ± 0.02 | 0.51 | 0.46 | 0.80 ± 0.22 |

### Example 3

### Antiviral Activity of Compound 20 Against Non-Cytopathic BVDV

MDBK cells were seeded into 6-or 96-well plates at a density of approximately 2×10⁴ cells per well. The cultures were incubated 3-24 hours to permit attachment of the cells to the plates prior to infection and addition of compounds. The appropriate number of non-cytopathic BVDV Pe515 (ncp BVDV) PFU were added to each well to achieve the desired MOI (0.001); the cells were contacted with the virus diluted at the appropriate concentration in PBS containing 1% HS and 1 mM MgCl₂ for 1 to 2 hours. The test compounds dissolved in cell growth media with 2% HS were added to the cells at varying concentrations. The plates were incubated at 37° C. in the presence of 5% CO₂ for 3-4 days. Uninfected cells and infected, untreated cells (without compound) were used as additional controls. The final volume was 100 μl per well. After 3-4 days of incubation, RNA was isolated from BVDV-containing supernatant using the QIAamp® viral RNA mini kit (as recommended by the manufacturer). Real-time quantitation was done using the Quantitative One Step Reverse Transcriptase (RT) Polymerase Chain Reaction (qRT-PCR) MasterMix Plus for SYBR® Green I (Eurogentec, San Diego, calif.). Real-time qRT-PCR data were collected on an Applied Biosystems Prism® 7300 SDS instrument controlled by a computer running version 1.0 of the 7300 SDS Collection software. The following primers were used for the one step RT-PCR SYBR quantitation assay: forward primer 5′-GCACCCCTGCTTGCTTACC-3′ (SEQ ID NO:1) and reverse primer 5′-CCATCCTCTGCGTTAGTATCAAACT-3′ (SEQ ID NO:2). The instrument was typically configured for the following run conditions: 25 μL sample volumes; one 30 minute 48° C. reverse transcription reaction, one 10 minute 95° C. polymerase activation step, followed by 40 cycles of 2-step qPCR (15 s of 95° C. denaturation, 60 s of 62° C. combined anneal/extension). Well-to-well variations in background fluorescence were corrected for by use of a ROX-labeled passive reference, included as part of the Eurogentec One Step qRT-PCR Master Mix for each sample. Amplification curves were analyzed by using empirically established cycle threshold and baseline settings for the assay. For each qPCR run, the SDS Collection software generated a linear calibration plot of C_{T} (threshold cycle) by using amplification results from a freshly prepared dilution series of pre-quantified BVDV RNA fragments generated using the Ambion MEGAshortscript® in vitro transcription kit (Austin, Tex.). A BVDV RT-PCR amplified DNA fragment using the forward primer 5′-TAATACGACTCACTATAGGGAACAAACATGGTTGGTGCAACTGGT-3′ (SEQ ID NO:3) and the reverse primer 5′-CTTACACAGACATATTTGCCTAGGTTCCA-3′ (SEQ ID NO:4), was used as a template for in vitro transcription. Control RNA was quantitated by measuring optical density, and copy number was calculated from the quantity and molecular weight of RNA. For the calibration curve, control BVDV-RNA was prepared in duplicate in 10-fold serial dilutions (10⁴-10¹¹ copies/mL). BVDV genomic RNA quantifications for unknown samples were interpolated from the resulting linear calibration curve. These calibration and interpolation steps are semi-automatic features of the SDS Collection software.

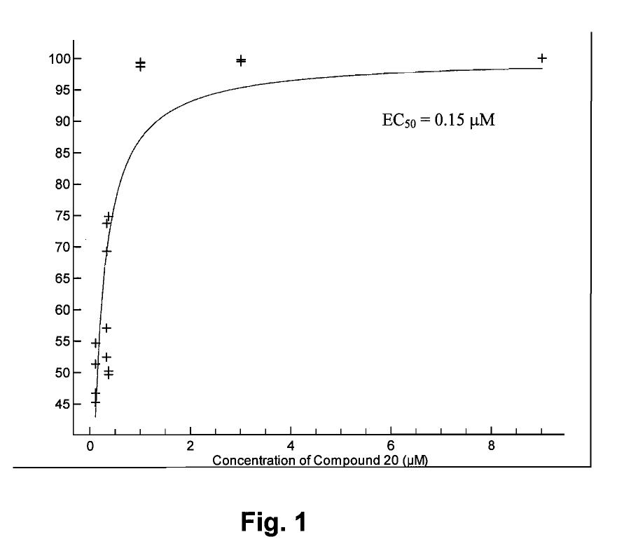

The data presented in FIG. 1 show that Compound 20 is a potent inhibitor of ncp BVDV genomic replication with an EC₅₀ of about 0.15 μM.

### Example 4

### Synergism of Compound 20 Combined with Interferon-α

A protection from BVDV cytopathic effect assay was performed to determine the potential of Compound 20 to act synergistically with IFN-α. A two-way and three-way combination assay was performed with average background-and color-corrected data in an inhibition-of-cytopathic effect (CPE) assay as described below.

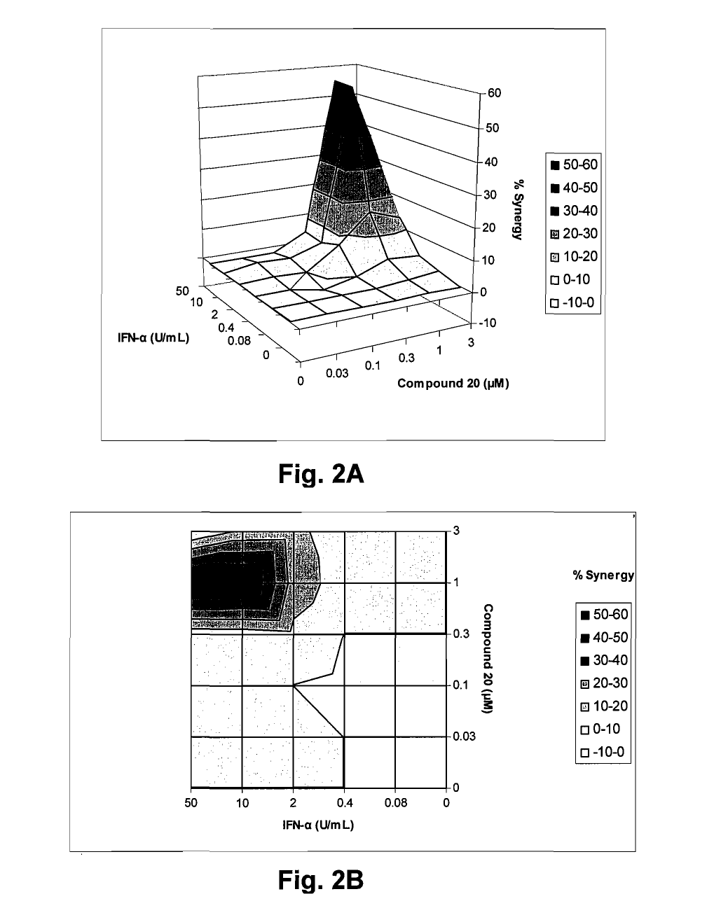

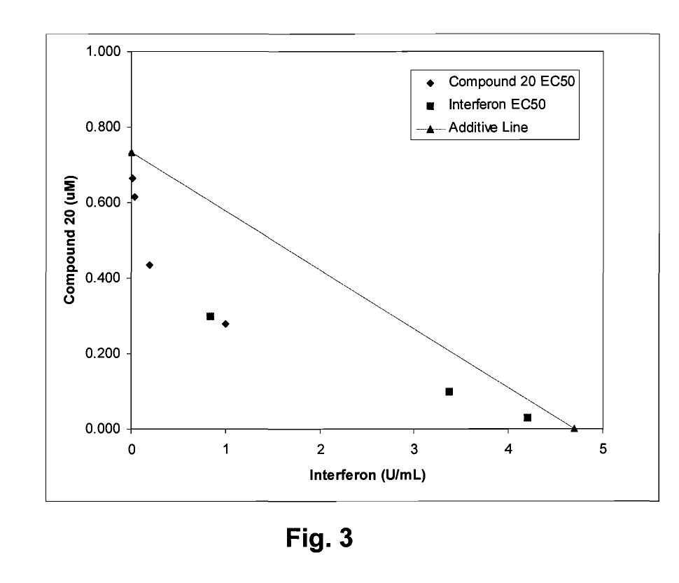

The checkerboard approach was used with various concentrations of IFN- [symbol] added to quantitate protection from BVDV-induced cytopathic effect on MDBK cells. The synergy volumes were analyzed using isobolograms (Suhnel (1990). Antiviral Res. 13: 23-39.) or MacSynergy II software (Prichard and Shipman, 1990; Prichard and Shipman, 1996). The isobologram indicates that Compound 20 has a synergistic interaction with IFN-α because the experimental EC₅₀ of Compound 20 or IFN-α falls below the theoretical additive value of these two compounds (FIG. 3). Three-dimensional (3-D) and 2-D graphs generated from the MacSynergy II software and representing the double synergy volumes of Compound 20 and IFN-α are presented in FIG. 2. A synergy volume of zero or close to zero represents additivity (volumes between −25 and 25 μM(IU/mL), negative volumes represent antagonism (Volumes<−25 IU/mL), and volumes>25 IU/mL represent synergism. The synergy volume of Compound 20 and IFN-α was measured to be 185.03 μM(IU/well)% (95% confidence interval, CI), which is considered strong and likely to be relevant in vivo.

### Example 5

### Cytotoxicity if INF-α Reduced by Tetracyclic Steroid-Like Compounds

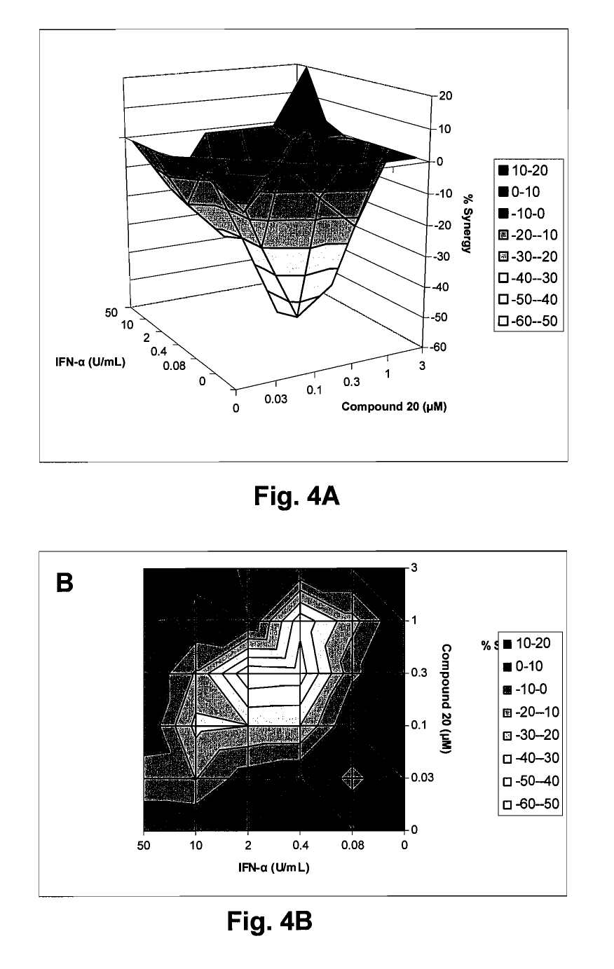

The purpose of this study was to evaluate the potential for synergy or antagonism cytotoxicity of Compound 20 with interferon-α2b (IFN-α). The checkerboard approach was used with various concentrations of Compound 20/IFN-α added to non-infected MDBK cells as described in Example 4. The synergy and antagonism volumes were analyzed using the MacSynergy II software (Prichard and Shipman, 1990; Prichard and Shipman, 1996). Three-dimensional and 2-D graphs generated from the MacSynergy II software and representing synergy/antagonism volumes of Compound 20 combined with IFN-α are presented in FIG. 4. The data presented in Table 5 represents the average synergy and antagonism volumes from duplicates obtained in one experiment. A synergy volume of zero or close to zero represents additivity (volumes between −25 and 25 μM (IU/mL), negative volumes represent antagonism (Volumes<−25 IU/mL), and volumes>25 IU/mL represent synergism. Additivity or antagonism between compounds in a cytotoxicity assay is an indication that the combination of compounds do not increase cytotoxicity (additivity) or show a reduction in cytotoxicity (antagonism). That is, the percentage value below the horizontal plane (additivity surface) reveals the regions and corresponding drug concentrations at which cytotoxicity is not increased and even possibly diminished (see FIG. 4). The combination of IFN-α and Compound 20 did not induce significant synergy in the cytotoxicity assay (Volume<25 μM (IU/mL)). The antagonism cytotoxic volume of IFN a combined with Compound 20 is considered strong (i.e., reduce cytotoxicity as compared to the compounds individually) and is probably relevant in vivo. The in vivo toxicity associated with the use of IFN-α or PEG-IFN may be reduced by administering the drug(s) in combination with compounds of structure (I) as described herein.

TABLE 5
Cytotoxicity of Double Combination of Compound 20/IFN-α

Cytotoxicity
Volume (95% CI)
μM(IU/mL) %

| Combination | Synergism | Antagonism |
|---|---|---|
| Compound 20 + IFN-α | 4.42 | −150.97 |

### Example 6

### Activity of Estrogens Against Hepatitis C Virus in a Sub-Genomic Replicon Assay

The antiviral activity of test compounds were assayed in the stably HCV RNA-replicating cell line, AVA5, derived by transfection of the human hepatoblastoma cell line, Huh7 (Blight, et al., 2000, Science 290: 1972). Compounds were added to dividing cultures once daily for three days (media is changed with each addition of compound) with 4-5 concentrations of test compound (2-3 cultures per concentration). Cultures generally start the assay at 50% confluence and reach confluence during the last day of treatment. Intracellular HCV RNA levels and cytotoxicity were assessed 24 hours after the last dose of compound.

Assays were conducted using 4-5 doses of test compound. A total of 4-6 untreated control cultures, and triplicate cultures treated with 10, 3, and 1 IU/ml interferon α (active antiviral with no cytotoxicity), and triplicate cultures treated with 100, 10, and 1 μM ribavirin (no antiviral activity and cytotoxic) served as controls. HCV RNA levels were assessed 24 hours after the last dose of compound using a dot blot hybridization assay. Both HCV and β-actin RNA levels in triplicate treated cultures were expressed as a percentage of the mean levels of RNA detected in untreated cultures (6 total). β-Actin RNA levels were used both as a measure of toxicity, and to normalize the amount of cellular RNA in each sample. Toxicity analyses were performed on separate plates from those used for the antiviral assays. Cells for the toxicity analyses were cultured and treated with test compounds with the same schedule and under identical culture conditions as used for the antiviral evaluations. Uptake of neutral red dye was used to determine the relative level of toxicity 24 hours following the last treatment. The absorbance of internalized dye at 510 nm (A₅₁₀) was used for the quantitative analysis. Values in test cultures were compared to 9 cultures of untreated cells maintained on the same plate as the test cultures. The results presented in Table 7 indicate that the compounds of structure (I), as described herein, are potent HCV antiviral agents with a favorable safety profile.

TABLE 6
Efficacy of the Compounds of Structure (I) Against
HCV in a Sub-Genomic Replicon Genotype 1b

| Compound # | EC₅₀ (μM) | CC₅₀ (μM) | SI |
|---|---|---|---|
| 16 | 0.510 | >100 | >196 |
| 20 | 0.043 | 8.1 | 188 |
| 4 | 0.068 | 24 | 353 |

SI: Selectivity Index

### Example 7

### Synergy of Estrogens in Combination with Interferon or NM107 Against BVDV

The inhibition-of-cytopathic effect (CPE) assay of Example 2 was used to analyze the interaction of estrogens or estrogen analogs combined with Interferon α-2b (PBL Biomedical Laboratories, Piscataway, N.J.), NM107 (Toronto Research Chemicals, Canada), or celgosivir. The compounds were combined at fixed molar ratios and serially diluted 2-fold in cell growth medium to examine a range of 6 fixed ratio combinations including those having about an equipotent antiviral dose to a combination in which one test compound was used at a sub-optimal level (e.g., sub-therapeutic). The corresponding monotherapies were conducted in parallel to these combination treatments (EC₅₀ values for the monotherapy treatments are provided in Table 7).

The protection against BVDV-induced cytopathic effect in MDBK cells (MOI of 0.01) by the combined test compound treatments was quantified and the test compound interactions (synergism, additivity or antagonism) were analyzed with the CalcuSyn™ program (Version 2.0, Biosoft, Inc., UK) to generate a Combination Index (CI) value, in which a CI value of 1 equals additivity. The following criteria were used: CI values above 1.45 indicated strong antagonism; CI values between 1.2 and 1.45 indicated moderate antagonism; values between 1.10 and 1.20 indicated slight antagonism; values between 0.90 and 1.10 are nearly additive; values between 0.85 and 0.90 indicated slight synergism; values between 0.7 and 0.85 indicated moderate synergism; values between 0.30 and 0.70 indicated good synergism; values between 0.10 and 0.30 indicated strong synergism; and values below 0.10 indicated very strong synergism. Table 8 shows that the double combination of Compound 2 with IFN α-2b or NM107 results in good synergism with CIs between 0.3 to 0.6. In this assay, the combination of compound 20 with celgosivir did not show synergy and was generally additive (CIs values between 1.0 and 1.2). The combination of celgosivir with compound 20 can still be beneficial since their additivity indicate that by adding one you can reduce the dose of the other to obtain a similar effect, or will increase the total antiviral effect when both are combined compared to a monotherapy.

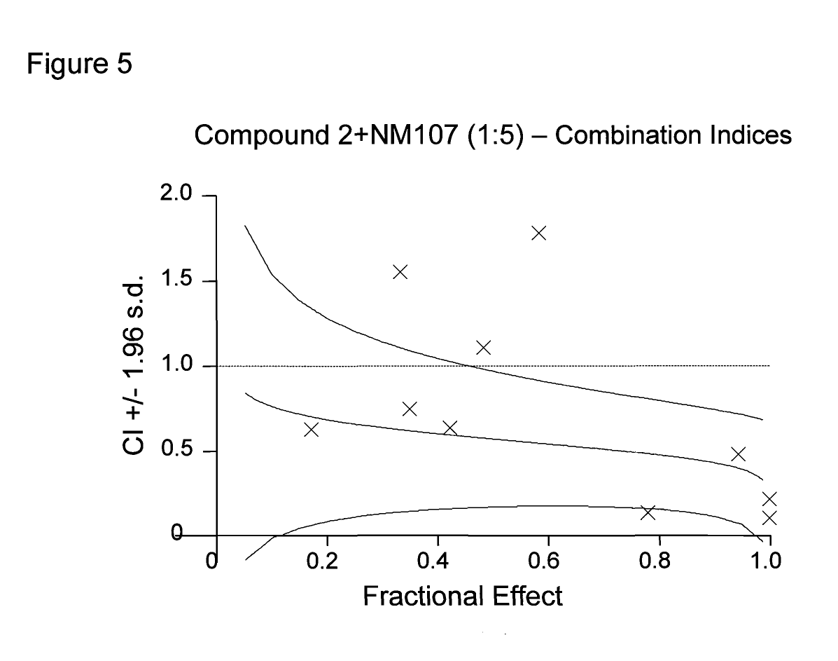

These values were plotted in Fraction of virus affected versus Combination Index plots (Fa—CI plots), which are generally the most useful in determining drug interactions because the Monte Carlo analysis provides a measure of statistical significance (i.e., these plots have three lines, which represent the median value (middle line) and ±1.96 standard deviations (upper and lower lines); see FIG. 5). The combination of Compound 2 with NM107 (FIG. 5) resulted in an Fa-CI plot that is indicative of synergy with the upper, median, and lower lines below 1 when greater than 40% of the viral fraction is affected, which shows that the synergy was highly significant. The CIs for the Compound 2 and NM107 combination were generally between 0.3 and 0.7 (medium line FIG. 5 and Table 8), indicating good synergism.

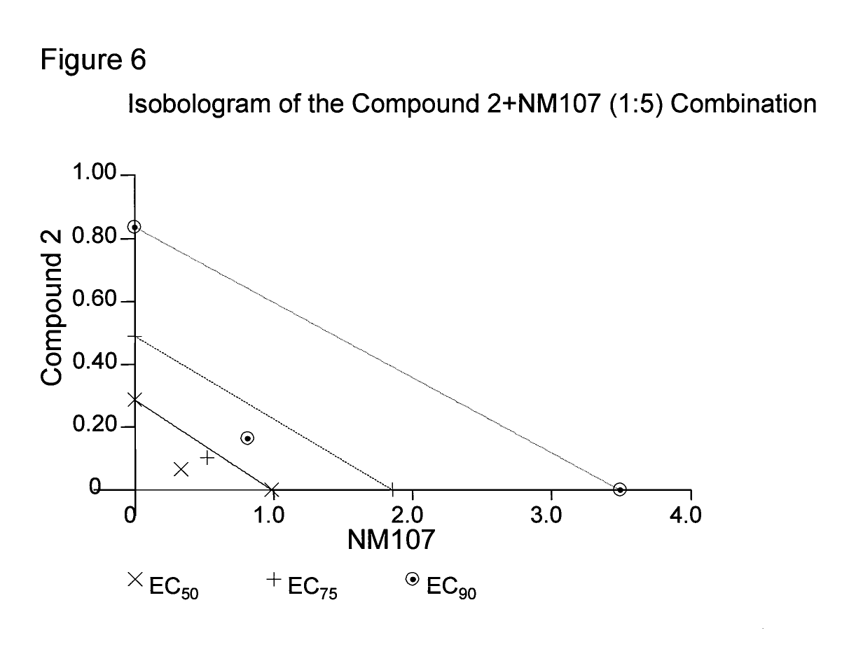

In addition, isobolograrns were generated, which provide an excellent secondary measure of the drug combination interactions. For these plots, EC₅₀, EC₇₅, and EC₉₀ values for the combination treatments are displayed as single points. Values that fell to the right of the additivity line (above) (i.e., the line drawn between the EC values for each drug as a monotherapy) indicated antagonism, values to the left of the additivity line (below) indicated synergy, and values on or near the line indicated additivity. See, for example, the combination of Compound 2 and NM107 in FIG. 6, in which the experimental EC₅₀s, EC₇₅s, and EC₉₀s were to the left side of their respective additivity line indicating synergy of the combination.

As is evident from Table 8 and FIGS. 5 and 6, the combination of Compound 2 with IFN-α, or the nucleoside analog NM107, was significantly synergistic.

TABLE 7
Monotherapy Potency of PPCs Obtained During Combination Studies

| | EC₅₀ | |
|---|---|---|
| Compound 2 | 0.23 | μM |
| IFN α-2b | 5.79 | IU/mL |
| NM107 | 0.99 | μM |
| Celgosivir | 2.2 | μM |

TABLE 8
Combination Indexes (CIs) of Various Estrogen or
Estrogen Analog Double Combinations

| Combination | Ratio | CI (EC₅₀) | CI (EC₇₅) | CI (EC₉₀) |
|---|---|---|---|---|
| Compound 2 + IFN α-2b | 1:30 | 0.576 | 0.437 | 0.341 |
| Compound 2 + NM107 | 1:5 | 0.569 | 0.493 | 0.428 |
| Compound 20 + Celgosivir | 1:25 | 1.143 | 1.080 | 1.027 |

As is evident from Table 8 and FIGS. 5 and 6, the combination of PPCs with IFN α or the nucleoside analog NM107 were synergistic.

### Example 8

### Activity of PPCs Against HIV

Testing of PPCs against HIV-1 was performed according to Weislow et al. (J. Natl. Cancer Inst. 81: 577-586, 1989) and Buckheit and Swanstrom (AIDS Res. Hum. Retrov. 7:295-302, 1991). Compounds 24, 20, 5, and 2 demonstrated activity in inhibiting HIV replication with therapeutic indices >1. Compound 16 showed some activity against HIV-1 in this assay (20% inhibition of HIV-1 induced cytotoxicity in CEM-SS cells at 300 μM and no cytotoxicity shown by the compound at that dose) (Table 9). The data in Table 9 indicate that PPCs can have significant activity against HIV.

TABLE 9
Anti-HIV-1 Activity of Various PPCs

| | | CEM-SS | Therapeutic |
|---|---|---|---|
| Compound | CEM-SS/HIV-1_{RF}EC₅₀ (μM) | CC₅₀ (μM) | Index |
| 16 | >300.0 | >300.0 | — |
| 24 | <0.95 | 12.4 | >13.1 |
| 20 | 8.6 | 14.3 | 1.7 |
| 5 | 5.8 | 17.3 | 3.0 |
| 2 | 19.7 | >100.0 | >5.1 |

Therapeutic Index = CC₅₀/EC₅₀

### Example 9

### Synthesis of a Representative Compound of Structure (I-b)

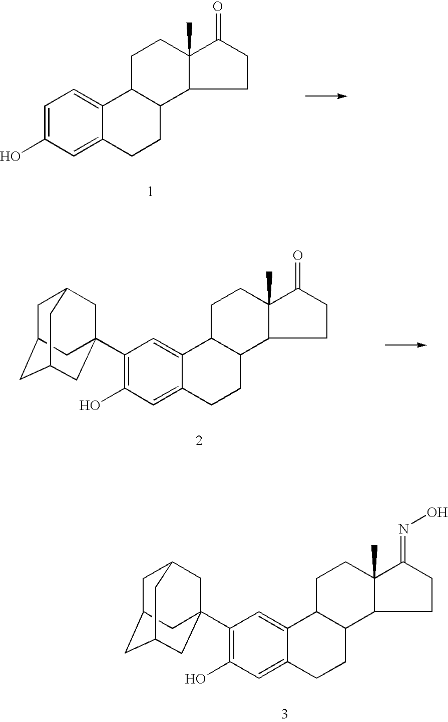 Synthesis of Compound 2

To a stirred suspension of estrone (compound 1) (1.08 g) and 1-adamantanol (0.70 g) in hexane (30 ml) at 0° C. under an argon atmosphere was added BF₃.Et₂O (1.6 ml) drop-wise via a syringe. The mixture was stirred and allowed to warm to room temperature over a period of 4 hours. The solvent was then removed under vacuum. The residue was titrated with water. The resulting solid was collected by filtration and re-crystallized from a mixed solvent of ethyl acetate and hexane to yield compound 2 as a white solid (0.792 g). ¹H NMR (CDCl₃) δ=7.14(s, 1H), 6.41(s, 1H), 4.68(s, 1H), 2.81(m, 2H), 2.48(m, 2H), 2.25(m, 2H), 2.0-10(m, 8H), 1.95(m, 2H), 1.77(s, 5H), 1.58(m, 6H), 1.43(m, 2H), 1.25(m, 1H), 0.91(s, 3H).

Synthesis of Compound 3

To a solution of compound 2 (0.202 g) in methanol (20 ml) was added hydroxylamine hydrochloride (0.350 g) and pyridine (2.0 ml). The mixture was refluxed for 17 hours. The mixture was then titrated with water. The resulting solid was collected by filtration and dried under high vacuum to yield compound 3 as a white solid (0.167 g). LC-MS calc'd. for C₂₈H₃₇NO₂: 419; found: 420 [M+1].

### Example 10

### Synthesis of Further Representative Compounds

Using the procedure set forth in Example 6 for the synthesis of compound 2, the following compounds were also prepared. 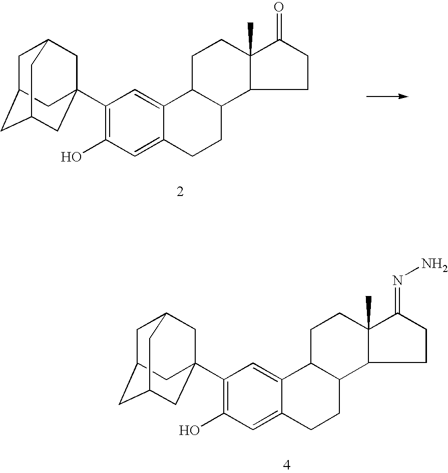
Synthesis of Compound 4

To a solution of compound 2 (0.202 g) in ethanol (10 ml) was added hydrazine (1.0 ml). The mixture was refluxed for 17 hours, and the solvent was then removed under vacuum. The residue was purified on a silica gel column using 2.5% methanol in dichloromethane to yield compound 4 as a white solid (0.172 g). LC-MS: calc'd. for C₂₈H₃₈N₂0: 418; found: 419. ¹H NMR (CDC1₃) δ=7.15(s, 1H), 6.40(s, 1H), 5.16(bs, 1H), 4.80(bs, 2H), 2.78(m, 3H), 2.50-1.20(m, 27H), 0.89(s, 3H). 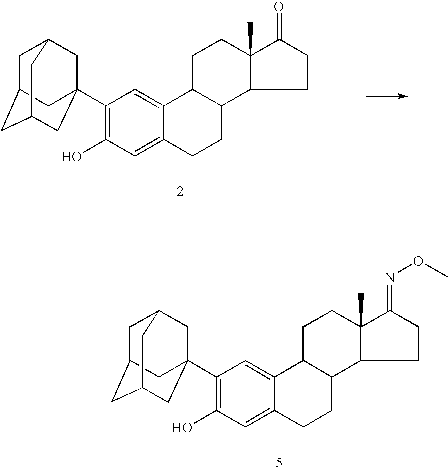
Synthesis of Compound 5

To a solution of compound 2 (0.202 g) in methanol (10 ml) was added O-methyl hydroxylamine hydrochloride (0.415 g) and pyridine (1.0 ml). The mixture was refluxed for 17 hours. The solvent was then removed under vacuum. The residue was dissolved in ethyl acetate and washed with brine. The crude product was purified on a silica gel column using 10% ethyl acetate in hexane to yield compound 5 as a pale yellow solid (0.197 g). LC-MS: calc'd. for C₂₉H₃₉NO₂: 433; found: 431. ¹HNMR (CDCl₃) δ=7.15(s, 1H), 6.40(s, 1H), 4.18(bs, 1H), 3.85(s, 3H), 2.78(m, 3H), 2.51-1.27(m, 27H), 0.94(s, 3H). 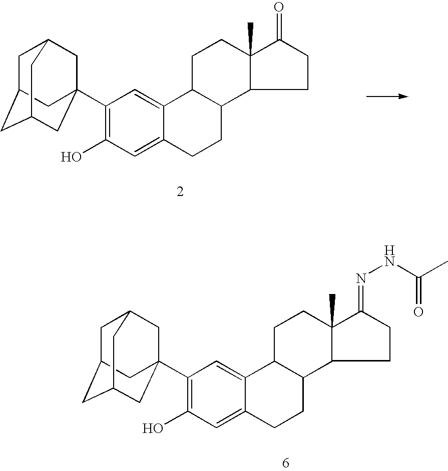
Synthesis of Compound 6

To a solution of compound 2 (0.100 g) in ethanol (10 ml) was added acetohydrazide (0.185 g). The mixture was refluxed for 18 hours. The mixture was then titrated with water. The resulting solid was collected by filtration, washed with water, and dried under high vacuum to yield compound 6 as a white solid (0.102 g). LC-MS: calc'd. for C₃₀H₄₀N₂O₂: 460; found: 483 (M+Na).

### Example 11

### Synthesis of Further Reprtsentative Compounds, Affinity Resins and Biotin Derivatives

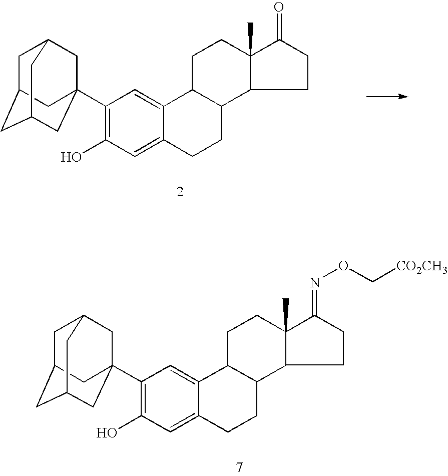 Synthesis of Compound 7

To a solution of compound 2 (0.202 g) in methanol (10 ml) was added carboxymethoxyl amine hemihydrochloride (0.370 g). The mixture was refluxed for 18 hours. The mixture was then diluted ethyl acetate and washed with saturated NaHCO₃ (aq.), brine, and then dried over sodium sulfate. The crude product was purified on a silica gel column using 20% ethyl acetate in hexanes as the eluent to give compound 7 as a white solid (0.198 g). ¹H NMR (CDC1₃) δ=7.13(s, 1H), 6.40(s, 1H), 4.83(bs, 1H), 4.61(s, 2H), 3.76(s, 3H), 2.77(m, 2H), 2.60(m, 2H), 2.39(m, 1H), 2.25(m, 1H), 2.10(m, 11H), 1.90(m, 3H), 1.63-1.36(m, 8H), 0.93(s, 3H). 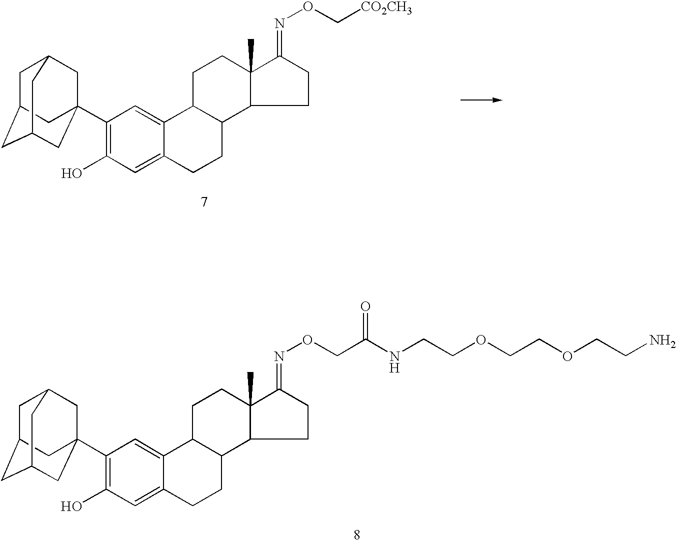
Synthesis of Compound No. 8

A solution of compound 7 (40 mg) and 2,2′-(ethylenedioxy)bis(ethylamine) (100 mg) in methanol (2 ml) was refluxed for 18 hours. The mixture was diluted with ethyl acetate and washed with water (5×) and dried over sodium sulfate to yield compound 8 as a white solid. The crude product was analytically pure. LC-MS: calc'd. for C₃₆H₅₃N₃O₅: 607; found: 608. 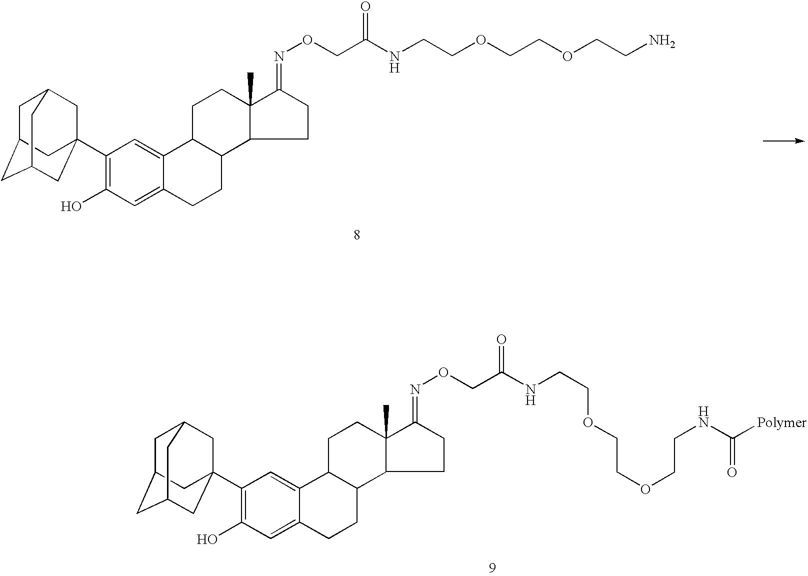
Synthesis of Affinity Resin No. 9

NHS activated-sepharose resin (20 ml) was washed with NMP (4×) and shaken with a solution of compound 8 (6.1 mg) in NMP (20 ml) and DIEA (1 ml) at room temperature for 4 hours. LC-MS indicated complete disappearance of compound 8 in the solution. Ethanolamine (1 ml) was added, and the mixture was shaken for 18 hours to yield affinity resin 9, which was then washed with methanol (3×), NMP (3×) and methanol (3×). 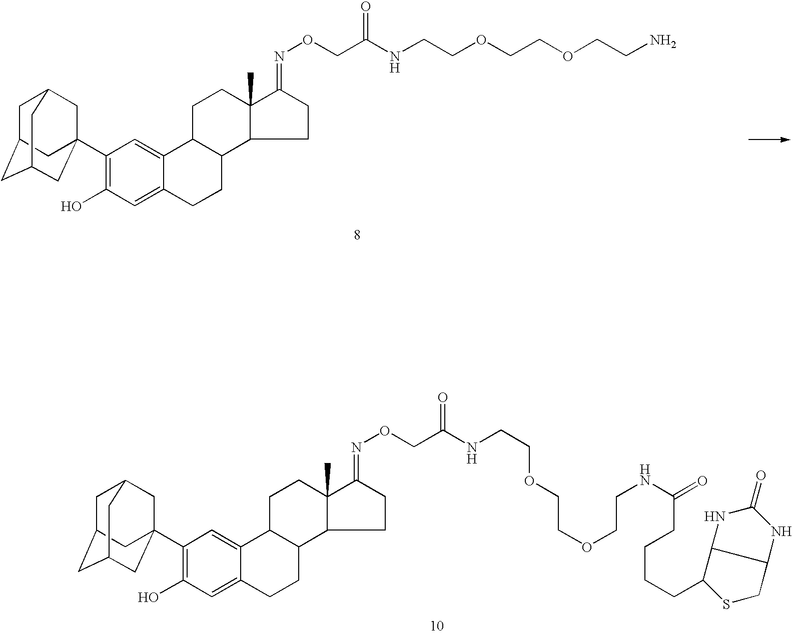
Synthesis of Biotin Derivative No. 10

Compound 8 (0.179 g, 0.29 mmole) was dissolved in DCM/DMF(1/1, 20 ml). DIEA (1 ml) and biotin-NHS ester (60 mg) were added. The mixture was stirred at room temperature for 18 hours. The solvent was removed under vacuum. The residue was purified on RP-HPLC to yield biotin derivative 10 as an off-white solid (0.123 g). LC-MS: calc'd. for C₄₆H₆₇N₅O₇S: 833; found: 834.

### Example 12

### Synthesis of Further Compounds

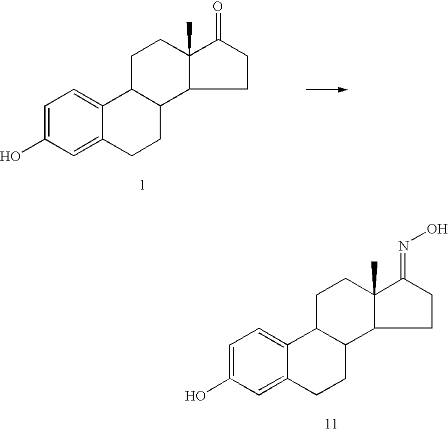 Synthesis of Compound No. 11

To a solution of estrone (compound 1) (0.540 g) in methanol (20 ml) was added hydroxylamine hydrochloride (0.690 g) and pyridine (2.0 ml). The mixture was refluxed for 17 hours. The mixture was then titrated with water. The resulting solid was collected by filtration and dried under high vacuum to yield compound 11 as a white solid (0.167 g). LC-MS: calc'd. for C₁₈H₂₃NO₂: 285; found: 286. ¹H NMR (CDCl₃) δ=9.14(bs, 1H), 7.90(s, 1H), 7.08(d, 1H), 6.58(d, 1H), 6.52(s, 1H), 3.31(s, 1H), 2.84(m, 3H), 2.46(m, 2H), 2.35(m, 1H), 2.20(m, 1H), 2.05(s, 1H), 1.93.(m, 2H), 1.50(m, 4H), 0.91(s, 3H). 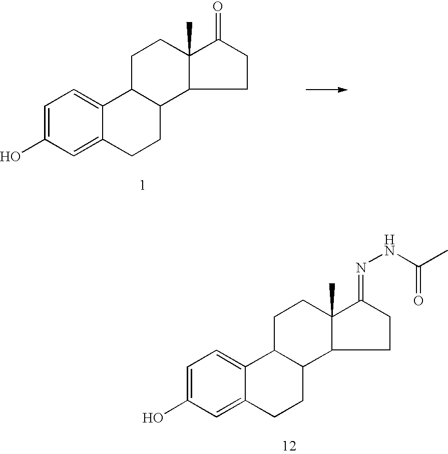
Synthesis of Compound 12

To a solution of estrone (compound 1) (0.540 g) in ethanol (20 ml) was added acetohydrazide (0.740 g) and pyridine (2.0 ml). The mixture was refluxed for 18 hours. The mixture was then titrated with water. The resulting solid was collected by filtration, washed with water and dried under high vacuum to give compound 12 as a white solid (0.616 g). LC-MS: calc'd. for C₂₀H₂₆N₂O₂: 326; found: 327. ¹H NMR showed a mixture of two rotamers. 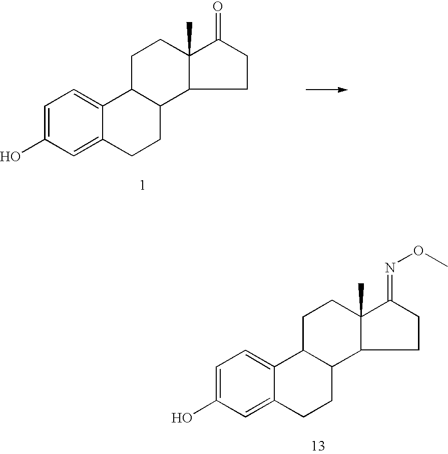
Synthesis of Compound 13

To a solution of estrone (compound 1) (0.540 g) in ethanol (20 ml) was added O-methyl-hydroxylamine (0.835 g) and pyridine (2.0 ml). The mixture was refluxed for 18 hours. The mixture was then titrated with water. The resulting solid was collected by filtration, washed with water and dried under high vacuum to give compound 13 as a white solid (0.576 g). LC-MS: calc'd. for Cl₉H₂₅NO₂: 299; found: 300. ¹H NMR (CDCl₃/DMSO) δ=8.25(s, 1H), 6.79(s, 1H), 6.31(s, 1H), 6.25(s, 1H), 3.51(s, 3H), 2.60(m, 3H), 2.25(m, 4H), 1.65(m, 3H), 1.18(m, 6H), 0.63(s, 3H). 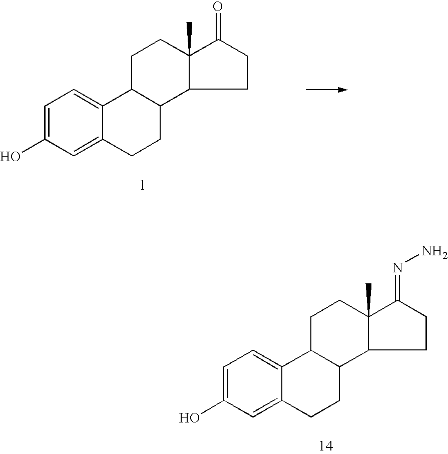
Synthesis of Compound 14

To a solution of estrone (compound 1) (0.540 g) in ethanol (20 ml) was added hydrazine monohydrate. The mixture was refluxed for 18 hours. The mixture was then titrated with water. The resulting solid was collected by filtration, washed with water, and dried under high vacuum to yield compound 14 as a white solid (0.552 g). LC-MS: calc'd. for C₁₈H₂₄N₂O: 284; found: 285. ¹H NMR (DMSO) δ=8.98(s, 1H), 7.04(d, 1H), 6.50(dd, 1H), 6.44(d, 1H), 5.33(s, 2H), 2.73(m, 2H), 2.25(m, 2H), 2.12(m, 2H), 1.85(m, 3H), 1.38(m, 5H), 1.22(m, 1H), 0.78(s, 3H). 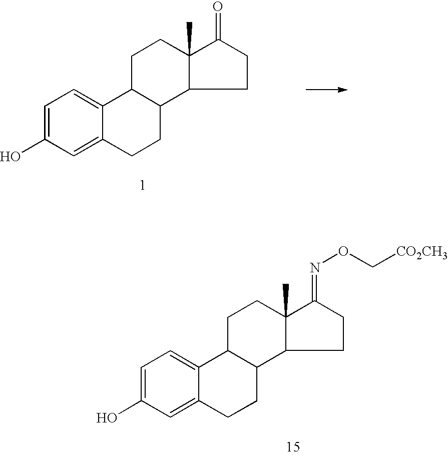
Synthesis of Compound 15

To a solution of estrone (compound 1) (0.540 g) in methanol (25 ml) was added carboxymethoxylamine hemihydrochloride (1.10 g). The mixture was refluxed for 17 hours. The mixture was then diluted with water. The resulting solid was collected by filtration and dried under high vacuum to give compound 15 as a white solid (0.700 g). ¹H NMR (CDC1₃) δ=7.13(d, 1H), 6.62(d, 1H), 6.56(s, 1H), 5.20(bs, 1H), 4.60(dd, 2H), 3.76(s, 3H), 2.82(m, 2H), 2.60(m, 2H), 2.00(m, 1H), 1.88(m, 2H), 1.59(m, 2H), 1.40(m, 6H), 0.93.(s, 3H).

### Example 13

### Synthesis of a Representative Compound of Structure (I-a)

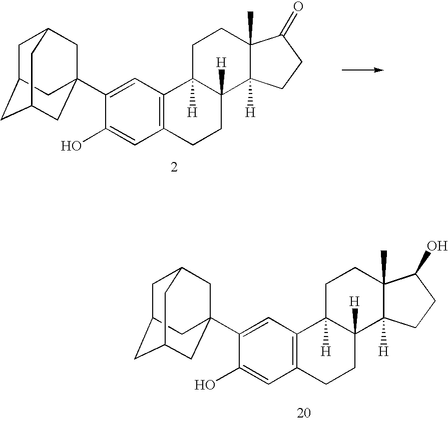

To a solution of compound 2 (250 mg, 0.62 mmol) in cold ethanol (25 mL) and methanol (10 mL) was added sodium borohydride (NaBH4, 140 mg) in one portion and the reaction was continued with stirring for 2 hours. Solvents were removed on a rotary evaporator and crushed ice was added. On standing overnight, the initially formed oil became a solid. The solid was filtered and washed with water until the filtrate was pH neutral. The solid was dried in a vacuum oven at 50° C. to give the crude compound 20 (0.25 g), which was purified by flash chromatography (silica gel eluted with 18% ethyl acetate in hexanes). The pure compound 20 (200 mg, 79.6%) was recrystallized from chloroform and hexanes to obtain crystals (150 mg), and was then characterized as follows: (i) melting point=174-175.degree. C.; (ii) .sup.1 H NMR (CDCl.sub.3, 300 MHZ) .delta.0.81 (s, 3H, C.sub.18 —CH.sub.3), 2.76 (m, 2H, C.sub.6 —CH.sub.2), 3.73 (t, 1H, C.sub.17 —H), 4.78 (s, 1H, C.sub.3 —OH), 6.38 (s, 1H, C.sub.4 —H), 7.16 (s, 1H, C.sub.1 —H); (iii) .sup.13 C NMR (CDCl.sub.3, 300 MHZ) .delta.10.95, 23.02, 26.33, 27.12, 28.77, 28.98 (3.times.C), 30.49, 36.54, 36.71, 37.01 (3.times.C), 38.89, 40.68 (3.times.C), 43.20, 44.22, 49.96, 81.99, 116.84, 124.06, 132.06, 133.83, 135.20, 152.36.

Those skilled in the art will recognize, or be able to ascertain, using no more than routine experimentation, many equivalents to the specific embodiments of the invention described herein. Such equivalents are intended to be encompassed by the following claims.

1. A method of treating or preventing a viral infection, comprising administering an effective amount of a compound having the following structural formula (I): 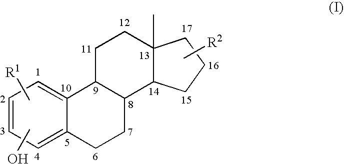
including stereoisomers, prodrugs and pharmaceutically acceptable salts or conjugates thereof, wherein:
R¹ is hydrogen, alkyl, substituted alkyl, aryl, substituted aryl, arylalkyl, substituted arylalkyl, carbocycle or substituted carbocycle;
R² is hydrogen, —OH, —O—R⁴, ═O, ═N—R³, —N(R¹⁴R¹⁴), —SH, —S—R⁴, or ═S;
R³ is alkyl, substituted alkyl, aryl, substituted aryl, arylalkyl, substituted arylalkyl, carbocycle, substituted carbocycle, heterocycle, substituted heterocycle, —N(R⁴R⁵), —O—R⁴, or —N(R⁴)C(═O)R⁵;
R⁴ and R⁵ are independently selected from hydrogen, alkyl, substituted alkyl, aryl, substituted aryl, arylalkyl, substituted arylalkyl, carbocycle, substituted carbocycle, heterocycle or substituted heterocycle; and
each R¹⁴ is independently hydrogen, alkyl, alkenyl, alkynyl, aryl, arylalkyl, arylheteroalkyl, arylaryl, heteroaryl or heteroarylalkyl.

2. The method according to claim 1 wherein R¹ is at position 2.

3. The method according to claim 2 wherein R¹ is alkyl.

4. The method according to claim 3 wherein the alkyl is tert-butyl.

5. The method according to claim 2 wherein R¹ is a carbocycle.

6. The method according to claim 5 wherein the carbocycle is adamantyl.

7. The method according to claim 1 wherein R² is at position 17.

8. The method according to claim 1 wherein R² is —OH(α) or —OH(β).

9. The method according to claim 1 wherein R² is ═N—R³.

10. The method according to claim 9 wherein ═N—R³ is ═N—NH₂ or ═N—O—CH₃.

11. The method according to claim 1 wherein the A ring —OH is at position 3.

12. The method according to claim 1 wherein the compound of structure (I) is 17(α)-estradiol or a conjugate thereof.

13. The method according to claim 12 wherein the conjugate is 17(α)-estradiol-3-sulfate sodium.

14. The method according to claim 1 wherein the compound of structure (I) is 17(β)-2-(1-adamantyl)-estra-1,3,5(10)-triene-3,17-diol.

15. The method according to claim 1 wherein the compound of structure (I) is 17(β)-2-(tert-butyl)-estra-1,3,5(10)-triene-3,17-diol.

16. The method according to claim 1 wherein the compound of structure (I) is compound 2, 3, 4, 5, or 20.

17. The method according to claim 1 wherein the viral infection is caused by a Flaviviridae, Hepadnaviridae, Herpesviridae, Adenoviridae, Papillomaviridae, Retroviridae, or respiratory virus.

18. The method according to claim 17 wherein the Flaviviridae is a hepatitis C virus.

19. The method according to claim 17 wherein the Retroviridae is human immunodeficiency virus (HIV)-1 or HIV-2.

20. A composition comprising a compound having the structural formula (I) according to claim 1 and an adjunctive antiviral therapy comprising:
a. a compound that inhibits viral infection of cells;
b. a compound that alters viral replication;
c. a helicase inhibitor;
d. a protease inhibitor;
e. an glucosidase inhibitor;
f. an inhibitor of an internal ribosome entry site (IRES);
g. a nucleoside analog;
h. a reverse transcriptase (RT) non-nucleoside inhibitor;
i. a compound that ameliorates the symptoms or effects of a viral infection; or
j. a compound that alters host immune function.

21. The composition according to claim 20 wherein the compound of structure (I) is 17(α)-estradiol or a conjugate thereof.

22. The composition according to claim 21 wherein the conjugate is 17(α)-estradiol-3-sulfate sodium.

23. The composition according to claim 20 wherein the compound of structure (I) is 17(β)-2-(1-adamantyl)-estra-1,3,5(10)-triene-3,17-diol.

24. The composition according to claim 20 wherein the compound of structure (I) is 17(β)-2-(tert-butyl)-estra-1,3,5(10)-triene-3,17-diol.

25. The composition according to claim 20 wherein the compound of structure (I) is compound 2, 3, 4 or 5.

26. The composition according to claim 20 wherein the compound that alters immune function is interferon.

27. The composition according to claim 26 wherein the interferon is pegylated interferon.

28. The composition according to claim 26 wherein the interferon is interferon-α.

29. The composition according to claim 27 wherein the interferon is interferon-α.

30. The composition according to claim 20 wherein the nucleoside analog is ribavirin, 2′-C-methyl cytidine or valopicitabine.

31. The composition according to claim 20 wherein the nucleoside analog is lamivudine (3TC), zidovudine (ZDV), azidothymidine (AZT), zalcitabine, dideoxycytidine, dideoxyinosine, emitricitabine (FTC), stavudine (4dT), didanosine, tenofovir disoproxil fumarate, adefovir dipivoxil, abacavir, abacivir sulfate, or any combination thereof.

32. The composition according to claim 20 wherein the compound that ameliorates the symptoms or effects of a viral infection is an antioxidant.

33. The composition according to claim 20 wherein the glucosidase inhibitor is castanospermine or derivative thereof.

34. The composition according to claim 20 wherein the castanospermine derivative is [1S-(1α,6β,7α,8β,8αβ)]-octahydro-1,6,7,8-indolizinetetrol 6-butanoate (celgosivir).

35. The composition according to claim 20 wherein the compound having the structural formula (I) and the adjunctive antiviral therapy are administered sequentially.

36. The composition according to claim 20 wherein the compound having the structural formula (I) is administered before the adjunctive antiviral therapy.

37. The composition according to claim 20 wherein the adjunctive antiviral therapy is administered before the compound having the structural formula (I).

38. The composition according to claim 20 wherein the compound having the structural formula (I) and the adjunctive antiviral therapy are administered concurrently.

39. The composition according to claim 38 wherein the compound having the structural formula (I) and the adjunctive antiviral therapy are admixed as a single composition and administered concurrently.

40. A method of treating or preventing a viral infection, comprising administering to a subject in need thereof an effective amount of a composition according to claim 20.

41. The method according to claim 40 wherein the subject is human.

42. The method according to claim 40 wherein the compound having the structural formula (I) according to claim 1 and the adjunctive antiviral therapy are administered by different routes.

43. The method according to claim 42 wherein the compound having the structural formula (I) according to claim 1 is administered orally.

44. The method according to claim 42 wherein the adjunctive antiviral therapy is interferon.

45. The method according to claim 44 wherein the interferon is part of an implanted device.

46. The method according to claim 40 wherein the compound having the structural formula (I) according to claim 1 and the adjunctive antiviral therapy are administered by the same route.

47. The method according to claim 40 wherein the viral infection is caused by a Flaviviridae, Hepadnaviridae, Herpesviridae, Adenoviridae, Papillomaviridae, Retroviridae, or respiratory virus.

48. The method according to claim 47 wherein the Flaviviridae is a hepatitis C virus.

49. The method according to claim 48 wherein the composition comprises the compound of structural formula (I) that is compound 2 and the adjunctive antiviral therapy that is interferon.

50. The method according to claim 48 wherein the composition comprises the compound of structural formula (I) that is compound 20 and the adjunctive antiviral therapy that is interferon.

51. The method according to claim 48 wherein the composition comprises the compound of structural formula (I) that is compound 2 and the adjunctive antiviral therapy that is NM107.

52. The method according to claim 48 wherein the composition comprises the compound of structural formula (I) that is compound 20 and the adjunctive antiviral therapy that is NM107.

53. The method according to claim 47 wherein the Retroviridae is human immunodeficiency virus (HIV)-1 or HIV-2.

54. The method according to claim 53 wherein the composition comprises the compound of structural formula (I) that is compound 2 and the adjunctive antiviral therapy that is an RT nucleoside inhibitor.

55. The method according to claim 54 wherein the RT nucleoside inhibitor is lamivudine (3TC), zidovudine (ZDV), azidothymidine (AZT), zalcitabine, dideoxycytidine, dideoxyinosine, emitricitabine (FTC), stavudine (4dT), didanosine, tenofovir disoproxil fumarate, adefovir dipivoxil, abacavir, abacivir sulfate, or any combination thereof

56. The method according to claim 54 wherein the composition further comprises a protease inhibitor.

57. The method according to claim 56 wherein the protease inhibitor is amprenavir (APV), tipranavir (TPV), saquinavir, saquinavir mesylate (SQV), indinavir, lopinavir, ritonavir (RTV), fosamprenavir calcium (FOS-APV), atazanavir sulfate (ATV), nelfinavir mesylate (NFV), darunavir, or any combination thereof.

58. The method according to claim 53 wherein the composition comprises the compound of structural formula (I) that is compound 20 and the adjunctive antiviral therapy that is an RT nucleoside inhibitor.

59. The method according to claim 58 wherein the RT nucleoside inhibitor is lamivudine (3TC), zidovudine (ZDV), azidothymidine (AZT), zalcitabine, dideoxycytidine, dideoxyinosine, emitricitabine (FTC), stavudine (4dT), didanosine, tenofovir disoproxil fumarate, adefovir dipivoxil, abacavir, abacivir sulfate, or any combination thereof

60. The method according to claim 58 wherein the composition further comprises a protease inhibitor.

61. The method according to claim 60 wherein the protease inhibitor is amprenavir (APV), tipranavir (TPV), saquinavir, saquinavir mesylate (SQV), indinavir, lopinavir, ritonavir (RTV), fosamprenavir calcium (FOS-APV), atazanavir sulfate (ATV), nelfinavir mesylate (NFV), darunavir, or any combination thereof.


SEQUENCE LISTING (4 sequences)

SEQ ID NO: 1 - DNA, 19 bp, artificial sequence
  primer
gcacccctgcttgcttacc

SEQ ID NO: 2 - DNA, 25 bp, artificial sequence
  reverse primer
ccatcctctgcgttagtatcaaact

SEQ ID NO: 3 - DNA, 45 bp, artificial sequence
  primer
taatacgactcactatagggaacaaacatggttggtgcaactggt

SEQ ID NO: 4 - DNA, 29 bp, artificial sequence
  reverse primer
cttacacagacatatttgcctaggttcca